(19) United States
(12) Patent Application Publication
(10) Pub. No.: US 2008/0097188 A1
(43) Pub. Date: Apr. 24, 2008

(54) EXTERNAL SENSING SYSTEMS AND METHODS FOR GASTRIC RESTRICTION DEVICES

(75) Inventors: Scott Pool, Laguna Hills, CA (US); Arvin Chang, West Covina, CA (US); Jay R. McCoy, Temecula, CA (US)

Correspondence Address: Vista IP Law Group LLP, 2040 MAIN STREET, 9TH FLOOR, IRVINE, CA, 92614, US

(73) Assignee: ELLIPSE TECHNOLOGIES, INC., Irvine, CA (US)

(21) Appl. No.: 11/779,818

(22) Filed: Jul. 18, 2007

Related U.S. Application Data
(63) Continuation-in-part of application No. 11/732,431, filed on Apr. 2, 2007.
(60) Provisional application No. 60/853,105, filed on Oct. 20, 2006.
(60) Provisional application No. 60/854,574, filed on Oct. 25, 2006.
(60) Provisional application No. 60/880,080, filed on Jan. 11, 2007.
(60) Provisional application No. 60/904,625, filed on Mar. 1, 2007.

(51) Int. Cl. A61B 5/05

(52) U.S. Cl. 600/409; 600/12; 606/153

(57) ABSTRACT

Methods and apparatus for quantifying the amount or rate of magnetically susceptible fluid within a gastric lumen are described. In one aspect, a magnetic sensor located external to the patient is configured to detect a quantity of fluid disposed within the gastric lumen. The quantity of fluid may include fluid that is contained upstream with respect to a restriction formed in the gastric lumen (e.g., by a gastric restriction device). The quantity of fluid disposed within the gastric lumen is determined by the magnetic sensor. This quantity may be evaluated over time to then calculate a real time flow rate which can then be displayed to the physician. The methods and devices allow a physician or other trained person to dynamically view real time development of fluid flow within a restricted gastric lumen and may be used in conjunction with adjustments to the gastric restriction device to achieve target or desired flow rates.

### RELATED APPLICATIONS

This Application is a continuation-in-part of U.S. application Ser. No. 11/732,431 filed on Apr. 2, 2007, which itself claims priority to U.S. Provisional Application Nos. 60/853,105 filed on Oct. 20, 2006, 60/854,574 filed on Oct. 25, 2006, 60/880,080 filed on Jan. 11, 2007, and 60/904,625 filed on Mar. 1, 2007. Priority is claimed to the above-noted Applications pursuant to 35 U.S.C. §§119, 120. In addition, each Application identified above is incorporated by reference as if set forth fully herein.

### FIELD OF THE INVENTION

Some embodiments of the present disclosure relate to apparatus and methods for monitoring and regulating gastrointestinal or other bodily restriction devices. In particular, some embodiments are directed to detecting a flow condition or determining a flow rate through such a device.

### BACKGROUND OF THE INVENTION

Obesity is an ever-increasing public health problem not only in the United States but in a number of other countries. In the U.S. it is estimated that more than 55% or nearly 100 million adults are overweight. Obesity can range from mild, to severe or morbid. The degree of obesity is typically characterized using a measure known as body-mass-index, or BMI. The BMI takes into account the individual's height and weight in order to establish a relative index of obesity. A normal BMI is considered to range from 18-25, while a BMI greater than 25 is considered overweight or obese. A BMI greater than 40 is considered morbidly obese.

It is well-established in the medical literature that obesity adversely affects general health, and can result in reduced quality of life and reduced lifespan. It is now well-accepted that obesity is associated with increased risk of cardiovascular disease, diabetes and other health issues. In contrast, animal studies show that longevity is increased in lean subjects (Weindruch, R. & Walford, R. L., 1988. The Retardation of Aging and Disease by Dietary Restriction, Thomas, Springfield, I L; Spindler, S. R., 2003, in Anti-Aging Therapy for Plastic Surgery, eds. Kinney, B. & Carraway, J., Quality Medical, St. Louis, Mo.).

TABLE 1
Risk of Associated Disease According to BMI and Waist Size

| | | Disease Risk | Disease Risk |
|---|---|---|---|
| | | Waist ≦40 in. | Waist >40 in. |
| | Weight | (men) or | (men) or |
| BMI | Classification | 35 in. (women) | 35 in. (women) |
| 18.5 or less | Underweight | — | N/A |
| 18.5 to 24.9 | Normal | — | N/A |
| 25.0 to 29.9 | Overweight | Increased | High |
| 30.0 to 34.9 | Obese Class I | High | Very High |
| 35.0 to 39.9 | Obese Class 2 | Very High | Very High |
| 40.0 to 49.9 | Morbidly Obese | Extremely High | Extremely High |
| >49.9 | Super Obese | Extremely High | Extremely High |

A number of approaches have been developed to deal with obesity as a means to improving individual health. The simplest method, dieting, can be effective but only if the individual adheres to a program of caloric restriction and exercise. Thus, even though dieting is relatively popular, many persons have difficulty in maintaining the long-term discipline needed for dieting to be an effective weight loss and weight maintenance regime. As a result, medical methods have been developed in order to assist people in losing weight and maintaining weight within normal ranges. Bariatrics is the branch of medicine concerned with the management of obesity and associated diseases. Several surgical methods have been developed that seek to effectively reduce caloric intake. These include procedures such as gastric bypass, gastroplasty, also known as stomach stapling and adjustable gastric banding.

In gastric bypass, a surgeon permanently changes the shape of the stomach by surgical reduction in order to create a smaller gastric pouch, or “new stomach”. The remainder of the stomach is then divided and separated from this pouch, thus reducing the amount of food that can be ingested. In addition, it is typical to bypass a portion of the small intestine, further reducing caloric uptake by reducing absorption in the gut. Once complete, this form of surgery is effectively irreversible.

In gastroplasty the surgeon staples the upper stomach to create a small pouch, with a capacity of about 1-2 ounces. A small stoma is created between the upper stomach pouch and the remainder of the stomach. No changes are made to the remainder of the digestive tract, and so this method is purely restrictive in nature.

A relatively less invasive procedure involves the use of an adjustable band to provide essentially the same result as a gastroplasty procedure, without the need to open the gastric cavity or perform any cutting or stapling operations. These bands are typically referred to in the literature as variously referred to interchangeably as an adjustable gastric restriction device or adjustable gastric band, or simply gastric band.

One such device is the Inamed Lap-Band®. This device is essentially an annular balloon that is placed around a portion of the stomach dividing the stomach into upper and lower pouches and creating a stomal opening between the two regions. The balloon is then inflated, typically with a saline solution, progressively closing the annulus around the stomach and reducing the size of the stoma between the upper and lower portions of the stomach. The first adjustment is usually performed several weeks after surgical placement of the gastric band, allowing time for the patient to heal, and for a fibrous tissue capsule to form around the band. The band can be inflated or deflated as necessary to alter the size of the stoma, thus providing at least in theory a method to tailor the device to each individual.

However, despite the advantages provided by gastric banding techniques, they nonetheless suffer from a number of drawbacks. The drawbacks include slippage, erosion, infection, patient discomfort and pain during the adjustment procedure, and an inability to determine the correct adjustment amount without using x-ray fluoroscopy with the swallow of a contrast solution to monitor rate of flow through the stomal opening.

Slippage may occur if a gastric band is adjusted incorrectly, for example, if the band is too tight. Slippage can also occur in response to vomiting, as occurs when a patient eats more food that can be comfortably accommodated in the upper pouch. During slippage, the size of the upper pouch may grow, causing the patient to be able to consume a larger amount of food before feeling full, thus lowering the effectiveness of the gastric band. During erosion, the gastric band migrates through the wall of the stomach, partially or completely contacting the stomach lumen. Though the etiology of erosion is not completely understood, some cases of erosion may occur if the gastric band is adjusted too tight, or if the stomach is sutured too tightly around the band. In either case, reducing the risk of slippage or erosion may be accomplished by adjusting the device to provide an appropriately sized stomal opening.

Infection and patient discomfort and pain are related to the use of the needle required to fill the gastric band with saline. As a result, non-invasively adjustable gastric bands have been proposed, some of which permit adjustment of the band without the need for invasive techniques such as needles. These bands also seek to provide a correct reading of the inner diameter of the gastric band at all times. However, because the wall thickness of the stomach is not uniform from patient to patient, the actual inner diameter of the stoma produced by the gastric band will be unknown. Thus the size of the opening of the band is at best an approximation of the stomal opening that connects the smaller upper pouch and the remainder of the stomach.

As a result, in order to properly monitor movement of material through the stoma, a means of determining flow condition or flow rate of ingested food through the stomach is required. Presently, no easy method exists for easily determining the flow rate through the stoma. Flow is typically monitored when the gastric band is adjusted, by tracking of a swallowed barium suspension by x-ray fluoroscopy. Examples of barium suspensions include Barosperse® and E-Z-Paque®.

The use of fluoroscopy presents its own problems. First, prior art methods of judging flow rate that make use of fluoroscopy require as part of the procedure exposure to x-rays. As x-rays are a form of ionizing radiation their use should always be with great consideration of the additional risks that radiation poses to humans. In certain patients the risk of radiation is increased. For example, a large percentage of the patients that receive gastric bands are women in the child bearing years. The few first weeks of pregnancy, when a mother may be unaware she is pregnant, is an especially critical time of fetal development and exposure to x-rays is to be avoided if at all possible.

In addition, in many centers, the use of x-ray fluoroscopy is cost-prohibitive, and often, the patient either lacks insurance coverage, or otherwise is unable to afford this kind of follow-up treatment. As an alternative, many centers do not use barium in combination with x-ray fluoroscopy but rather have the patient simply drink a quantity of water, for example cold water, which is more readily sensed by the patient. If the water does not pass, the gastric band is loosened. However, using this method, it is impossible to determine with any precision as to how tight or loose the band might be, other than in the most qualitative of sense that there is either an opening or there is not. In addition, even though water passes through the opening, the band may still be too tight to permit solid food to pass leading to patient discomfort and an increased risk of vomiting. The relatively high stresses imposed by vomiting increase the risk of movement or slippage of the band, in addition to increasing the patient's level of discomfort and anxiety.

Another perplexing factor is the fact that sometimes, the gastric band displays a diurnal variation. For example, the device may be tighter in the morning and looser in the evening. When adjustments are performed, it is not possible to know beforehand whether an initial adjustment of the opening produced by the band will be an optimal one. Consequently, depending upon what time of day the gastric band is placed and adjusted varying results may be seen in terms of flow of contents past the restriction. As well, more serious complication can arise from improper adjustment. For example, if the stomal opening produced by a band that is initially adjusted and considered to be adjusted correctly subsequently becomes blocked, such that even water fails to pass, the patient is in danger of quickly becoming dehydrated, a dangerous situation that may require emergent care.

While the use of barium suspension allows for visualization of the movement of material through the stomal opening, and provides a quantifiable method of adjustment, barium suspensions as typically used (e.g. 66% barium sulphate by weight in water) are many times more viscous than water. Barium suspensions also exhibit Non-Newtonian flow properties, making movement characteristics more difficult to predict. Even at reduced concentrations (e.g. 25% barium sulphate by weight in water) the solution is still 15 to 20 times as viscous as water. Even where certain barium sulphate suspensions are used that have a viscosity closer to that of water, for example Barosperse®, the suspension nonetheless may still exhibit Non-Newtonian flow behavior. Where the gastric band produces a very small stomal opening, viscous solutions may fail to flow through the opening.

Different patients require different degrees of restriction, depending on their eating habits, motivation, and other factors. Thus, at times it is desirable to adjust a gastric band to produce a very small stomal opening in order to achieve optimal weight control results. However, with very small openings, the viscosity of the barium suspension may not permit reliably detectable flow, and thus the restriction may be adjusted to provide a larger stoma than would be optimal in the particular case. It is also recognized that drinking barium suspensions is not pleasant to the patient due to the taste and texture of the material. Barium is also known to cause diarrhea in some individuals.

Alternative radio-opaque solutions are available that are iodine-based, for example Gastrografin®. Gastrografin® has a reported viscosity of 18.5 cP at 20° C. and 8.9 cP at 37° C. Consequently, as with barium suspensions, this is several times the viscosity of water, and in lower viscosity dilutions, the visibility using X-ray fluoroscopy is reduced. There is also an added risk in that some patients are allergic to iodine-based contrast agents such as Gastrografin®. Intravascular administration of iodine-based contrast agents is contraindicated in patients with compromised renal function, although additional laboratory testing for circulating creatinine levels (and added expense) are needed to confirm this. Rarely, vicarious renal secretion of contrast is observed in patients who have been given oral contrast agents. Thus, the use of all contrast solutions, whether barium-based, iodine-based or others, entails additional cost and risk.

### SUMMARY OF THE INVENTION

In one aspect of the invention, a magnetic sensor located external to the patient is configured to detect a quantity of fluid disposed within the gastric lumen. The quantity of fluid may include fluid that is contained upstream with respect to a restriction formed in the gastric lumen (e.g., by a gastric restriction device). The quantity of fluid disposed within the gastric lumen is determined by the magnetic sensor. This quantity may be evaluated over time to then calculate a real time flow rate which can then be displayed to the physician. The methods and devices allow a physician or other trained person to dynamically view real time development of fluid flow within a restricted gastric lumen and may be used in conjunction with adjustments to the gastric restriction device to achieve target or desired flow rates.

In another aspect of the invention, a method for determining the flow rate of a fluid passing through a restricted portion of a gastric lumen of a patient includes providing a magnetic sensor external to the patient, the magnetic sensor configured to detect a quantity of fluid disposed upstream of the restricted portion of the gastric lumen. The quantity of fluid disposed upstream of the restricted portion of the gastric lumen is measured at a first time with the magnetic sensor. The quantity of fluid disposed upstream of the restricted portion of the gastric lumen is measured at a second time with the magnetic sensor. The flow rate is then determined by subtracting the quantity of fluid disposed upstream of the restricted portion of the gastric lumen at the second time from the quantity of fluid disposed upstream of the restricted portion of the gastric lumen at the first time and dividing the subtracted value by the elapsed time between the first time and the second time.

In another aspect of the invention, a method of adjusting a gastric restriction device that is configured to restrict a portion of a gastric lumen of a patient is provided. The method includes administering a volume of test fluid to a patient, the test fluid comprising a carrier fluid and a magnetically detectable component. The flow rate of the test fluid through the restriction of the gastric lumen is measured using a magnetic sensor device located external to the patient. The gastric restriction device is then adjusted to achieve a target flow rate through the restriction.

In yet another embodiment of the invention, a method of determining the quantity of fluid within a gastric lumen of the patient includes administering a quantity of test fluid to a patient, the test fluid comprising a carrier fluid and a magnetically detectable component. The quantity of test fluid contained in the gastric lumen is then measured using a magnetic sensor device located external to the patient.

In still another embodiment of the invention, a system for determining the flow rate of fluid passing through a restricted portion of a gastric lumen of a patient includes at least one transmit coil operatively coupled to an alternating current source and at least one receive coil configured for detecting a quantity of a magnetically detectable fluid contained in the gastric lumen. The at least one receive coil is operatively coupled to a controller configured to output a signal corresponding to the quantity of magnetically detectable fluid contained in at least a portion of the gastric lumen over a plurality of times.

In yet another embodiment of the invention, a system for ascertaining flow of fluid passing through a restricted portion of a gastric lumen of a patient includes a test fluid comprising a carrier fluid and a metal and a metal detector configured to output a signal corresponding, at least in part, to flow of the test fluid through the restricted portion of the gastric lumen.

### BRIEF DESCRIPTION OF THE DRAWINGS

FIG. 1 illustrates a sectional view of the esophagus and stomach of a gastric restriction device patient undergoing a barium flow evaluation.

FIG. 2 illustrates a sectional view of the esophagus and stomach of a gastric restriction device patient undergoing barium flow evaluation.

FIG. 3 illustrates a sectional view of the esophagus and stomach of a gastric restriction device in a patient where the stomal opening is closed in order to view the upper stomach pouch.

FIG. 4 illustrates a sectional view of the esophagus and stomach of a gastric restriction device in a patient where the device has slipped from its initial placement location.

FIG. 5 illustrates a view of an embodiment for detecting a sound producing fluid.

FIG. 6 illustrates a section view of an embodiment, where an acoustic capsule is used.

FIG. 7 illustrates a sectional view of an embodiment, where an effervescent solution and inactivating solution are used.

FIG. 8A illustrates a view of an embodiment, using Doppler ultrasound detection of fluid movement in the stomach.

FIG. 8B shows a schematic of the principles underlying measurement of fluid velocity by Doppler ultrasound.

FIG. 9 is a sectional view of an embodiment, where scattering agents are include in the test substance.

FIG. 10A illustrates Doppler ultrasound recording data obtained from a patient.

FIG. 10B illustrates a spectral analysis of a sound recording from a Doppler ultrasound test in patient.

FIG. 11 depicts a wide array ultrasound probe, and strap for securing the probe to a patient.

FIG. 12 illustrates an embodiment that provides automated adjustment of the stoma based on acoustic feedback.

FIG. 13 is a graph of results of in vitro flow testing showing the time taken for 50 mL of a test substance to move past a simulated restriction.

FIG. 14 is a graph of results of in vitro flow testing showing the flow rate past a simulated restriction.

FIG. 15 illustrates one embodiment of a system for determining the flow rate of fluid passing through a restricted portion of a gastric lumen.

FIG. 16 illustrates another embodiment of a system for determining the flow rate of fluid passing through a restricted portion of a gastric lumen.

FIG. 17 illustrates the single transmission coil and two receive coils illustrated in FIG. 16.

FIG. 18 illustrates another embodiment of a system for determining the flow rate of fluid passing through a restricted portion of a gastric lumen.

FIG. 19 schematically illustrates a mass (m) of fluid disposed between respective sets of coils such as that illustrated in FIG. 18.

FIG. 20 schematically illustrates control electronics configured to drive multiple transmission and receive coils according to one aspect of the invention. FIG. 20 also illustrates a processor, optional external adjustment device, and display operatively coupled to the control electronics.

FIG. 21 illustrates a graph of measured flow rate as a function of time that may be generated as a gastric restriction device is changed from a fully closed state to an open state.

### DETAILED DESCRIPTION OF THE ILLUSTRATED EMBODIMENTS

As used herein, the term “gastric restriction device” is meant to include, without limitation, gastric bands, as well as any other device that can be used to restrict the lumen the stomach.

As used herein, the term “gastric lumen” is meant to include, without limitation, the entire lumen within a stomach, including any stomal opening produced by a gastric restriction device.

As used herein, the term “flow” is meant to include, without limitation, the ordinary meaning of the word flow, and in addition flow rate and flow condition, i.e. the presence or absence of flow.

As used herein, the term “sound-producing” is meant to include, without limitation, sound produced by a test substance related to its movement and can further include, without limitation, sound produced by flow, turbulent flow, cavitation, as well as sound reflection arising at an interface between a test substance and another substance or substances, whether it be due to cavitation of the test substance, or on the basis of differences in density or acoustic impedance between test substance and another substance or substances.

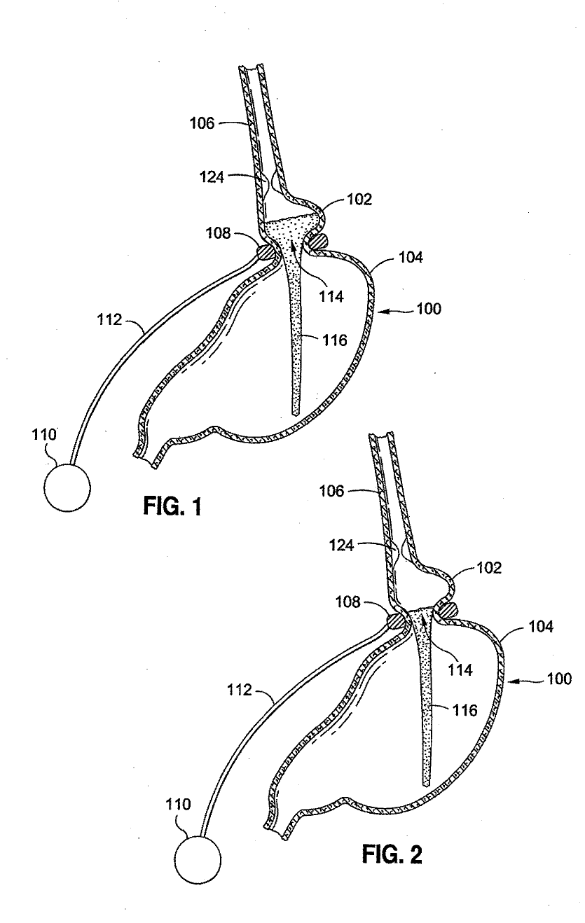

FIG. 1 illustrates a method of monitoring a gastric restriction device. The methods and devices of embodiments of the inventions described herein can be used with other lumenal restriction devices, such as those placed elsewhere in or around other regions of the gastrointestinal tract, such as the esophagus. The methods and devices can also be used with lumenal restriction devices used outside the gastrointestinal tract, such as in or around the bladder, urethra, ureters, vagina, uterus, fallopian tubes, seminal vesicles, bile ducts, pancreatic duct, etc. Also, as used herein, the term “gut” has its ordinary meaning and includes, without limitation, the alimentary canal (or the gastro-intestinal tract) from the mouth to the anus. During the monitoring method, the patient undergoes a visual flow evaluation test using barium contrast suspension 116 and X-ray fluoroscopy. The barium contrast solution 116 is radiopaque and is visualized using x-ray radiography. A gastric restriction device 108 is placed around the stomach 100, separating the stomach into an upper stomach pouch 102 and a lower stomach pouch 104. The gastric restriction device 108 is adjustable by means of an implantable interface 110. A dynamic change imparted to the implantable interface is transferred to the gastric restriction device via a line 112.

While being viewed by X-ray fluoroscopy, the barium contrast suspension 116 is ingested by the patient, passes down the esophagus 106, through the lower esophageal sphincter 124 and into the upper stomach pouch. The upper pouch 102 empties into the lower stomach pouch 104, through the stomal opening 114 produced by the gastric restriction device. FIGS. 1 and 2, respectively, depict the stomach and contents before and after a specific volume of barium suspension passes through the stomal opening.

In accordance with the present disclosure, possible configurations for the implantable interface 110 include, but are not limited to, an injection port, an inductive coupling, a sonically activatable coupling, a magnetic coupling (consisting of permanent magnets and/or electro-magnets), or a compressible pressurization member (such as a diaphragm and valve system). In some embodiments, configurations for the line 112 include, but are not limited to a fluid carrying tube, electrical conductors, a tension/compression cable-in-sheath system and a drive shaft-in-sheath system. Such variations of gastric restriction devices are compatible with the disclosure as described herein. In some embodiments, the dynamic change can be imparted directly to the gastric restriction device 108, eliminating the need for the implantable interface 110 and the line 112.

By knowing the initial volume of the barium contrast solution that was ingested, and by measuring the time for the upper stomach pouch to empty, the flow rate through the stoma opening can be calculated to be:

Mean Flow rate=(Volume of Barium Ingested Time to Empty) Eq. 1

For example, for a 10 mL to 75 mL room temperature bolus of barium sulphate suspended in water, an exemplary target mean flow rate is about 1 mL per second to about 20 mL per second. It should also be noted that this is an exemplary flow rate. More specifically, an exemplary target flow rate would be from about 5 mL to about 15 mL per minute when using a 50 mL volume of a standard Barosperse® suspension in water at room temperature.

Accounting for the viscosity of the barium suspension 116, the effective diameter of the stomal opening 114 can be calculated. As the level of barium suspension 116 in the upper stomach pouch decreases, so too will the hydrostatic pressure that drives movement of the barium suspension 116 through the stomal opening 114. The barium suspension 116 can be warmed to body temperature prior to sipping, so that there is no significant viscosity variation due to warming after ingestion, in turn making the stomal opening diameter calculation more straightforward to perform.

In the flow rate equation above, the mean flow rate is described. Note that as the upper pouch empties, the absolute flow rate decreases as the fluid level (and thus driving pressure) decreases. For a given stomal opening size, it is expected that the mean flow rate will be at least in part related to the initial volume of the bolus ingested. In some embodiments, residence time of the fluid in the upper stomach pouch might be a desirable measurement target, instead of mean flow rate or absolute flow rate. For example, where the restriction device provides an appropriate size opening, 30 mL of fluid would be expected to empty from the upper pouch in about four to six seconds.

It should also be noted that the restriction of the stoma may be affected in part by the width of the gastric restriction device 108, which in turn affects the length of the stoma. Some gastric restriction devices have starting widths varying from less than 14 mm to as wide as 23 mm. However, when restricted, many devices have an effective width that is less than the starting width, for example due to bowing of the balloon wall upon inflation, as can occur with a hydraulically actuated device.

Note that there is often variance in the effectiveness of a certain sized stomal opening from patient to patient. Whether a restriction device is providing the desired effect is typically a subjective determination based on patient feedback and in some cases observation by a caregiver. Different factors can affect the usefulness of the restriction device. These include among other things, a patient's own motivation to lose weight, a patient's tolerance to hunger and the quality of communication between the patient and their caregiver.

In addition, different patients may respond differently to a particular stomal opening size, and thus the most effective opening is likely to vary from patient to patient. For example, the most effective gastric restriction device internal diameter for weight loss may be 20 mm in one patient and 23 mm in another. Patient feedback as interpreted by a caregiver is one way in which stomal opening effectiveness is assessed. Patient feedback may include the amount of food that is eaten before the patient feels full, and the extent of vomiting that occurs if a patient consumes more food than the upper stomach pouch can reasonably hold. However, neither patient feedback nor caregiver observations are necessarily accurate measures of restriction device function. The present disclosure provides a needed improvement to gastric restriction devices in providing more precise measuring of flow rate past the restriction device to better tailor the patient's therapeutic regimen with their weight loss goals.

Traditionally, gastric band adjustments are performed or supervised by a bariatric surgeon. However, it is expected that by combining a non-invasive gastric restriction device adjustment means, with the reliable method of flow detection provided by the present disclosure, a non-physician may at least perform flow testing, and perhaps even the adjustment procedure.

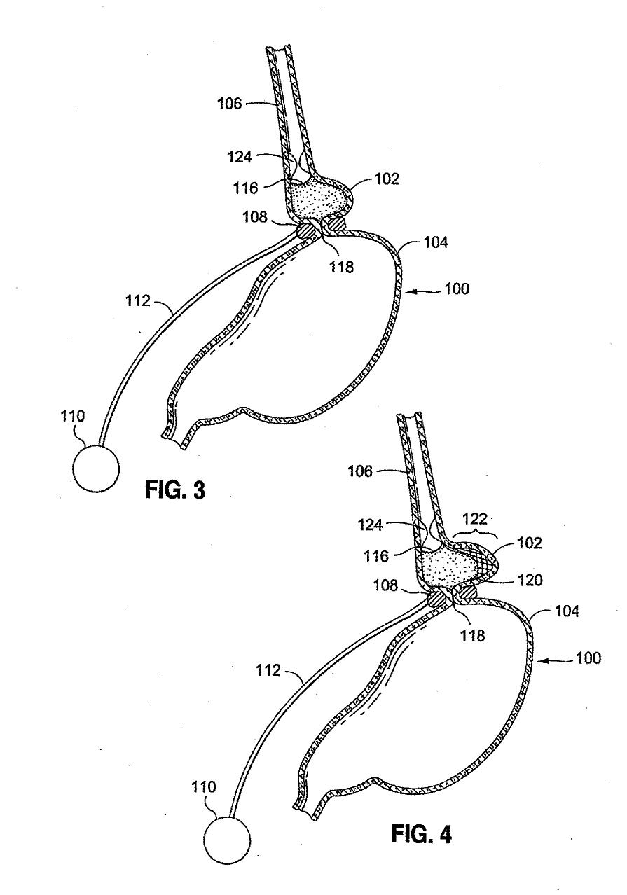

FIG. 3 illustrates a method of measuring the volume of the upper pouch 102, in order to determine whether any slippage of the device or upper stomach pouch growth has occurred. The gastric restriction device 108 is adjusted via the implantable interface 110 and the line 112 so that an occluded stoma 118 is created, and the patient's flow is effectively blocked. The patient now sips barium suspension in small gradations, for example, by drinking quantities of 10 mL until the upper stomach pouch is seen to be full on X-ray, for example when the upper level of the barium contrast solution is close to the lower esophageal sphincter 124. By knowing the total volume required to fill the upper stomach pouch 102, the general condition of the upper stomach pouch can be determined.

FIG. 3 illustrates an upper stomach pouch 102 that is at a desired volume. FIG. 4 illustrates an upper stomach pouch 102 that has grown undesirably, due to slippage of the stomach 100 relative to the gastric restriction device 108. The area of slippage 120 translates into an enlarged portion 122 of the upper stomach pouch 102. The volume of the pouch obtained from the barium study can be correlated with the size of the radio-opaque area as observed by fluoroscopy.

Using these methods, the stability of the gastric restriction device and its placement on the stomach can be monitored from one adjustment procedure to the other. By combining this information with the comments from the patient, a desirable setting for the gastric restriction device can be determined. For example, the gastric restriction device 108 may need to be tightened (to create a smaller stomal opening), loosened (to create a larger stomal opening), or the gastric restriction device 108 may need to be repositioned or removed. As described above, the barium swallow method can provide quantitative assessment of the stomal opening flow rate and the condition of the upper pouch.

All of the methods described so far require the use of radiographic procedures such as fluoroscopy in order to either measure the volume of the upper stomach pouch, or to monitor flow rate or residence time of material in the upper stomach pouch. In addition, these methods are further limited in that they are only useful to follow materials that are detectable by radiographic methods. Also, the contrast suspensions, having significantly higher viscosities than water, do not demonstrate a quantifiable flow where the stomal opening of a very small aperture, and so it may not be possible to accurately adjust the gastric restriction device to produce a very tight stomal opening, should that be desired.

In contrast, some embodiments of the invention provide alternative apparatus and methods to monitor and adjust the effectiveness of a gastric restriction device that reduce or avoid the use of X-ray fluoroscopy, and which are adapted for use with invasive or non-invasive means of adjusting a restriction device. These methods provide the further advantage in that they are non-invasive, involving the use of externally located monitoring means, and simple enough for a patient or caretaker to perform the testing procedure. This simplifies and reduces the cost of testing, and enhances patient involvement in achieving their weight loss goals.

The disclosure further provides methods of adjusting and monitoring the status of a gastric restriction device. In some embodiments the disclosure provides a non-invasive means of measuring flow through the stomal opening, or determining residence time in the upper stomach pouch. In some embodiments the method includes administering to a patient a known volume of a test substance detectable by a non-radiographic method, using a sensor means to detect the presence of the fluid at, or near, the stomal opening, producing an output from the sensor, and using the output signal from the sensor to monitor passage of the test substance through the stomal opening. From this, one can determine a flow condition, and if desired, by determining the time it takes for known volume of the test substance to move through the stomal opening, a flow rate can be calculated. As used herein, the term “flow condition” refers, without limitation, to the qualitative determination of whether there is flow or no flow through the stomal opening produced by a gastric-restriction device. The term “flow rate” refers, without limitation, to a calculation of flow in terms of an average volume per unit time of a test substance through the stomal opening.

### Sound Detection

In the present disclosure, sound can be advantageously used to monitor flow of a test substance past a gastric restriction device. In some embodiments described herein the test substance is exemplified as a fluid, preferably a liquid, which is detectable by non-radiographic methods. However, the disclosure does not necessarily depend on the test substance comprising a fluid, although in many cases it will be more convenient to use one. As a result, the disclosure is not intended to be limited to the use of fluids alone in practicing the invention as claimed, and any suitable substance that is compatible with the methods and apparatus disclosed herein is intended to fall within the scope of the term “test substance” as the term is used in this disclosure.

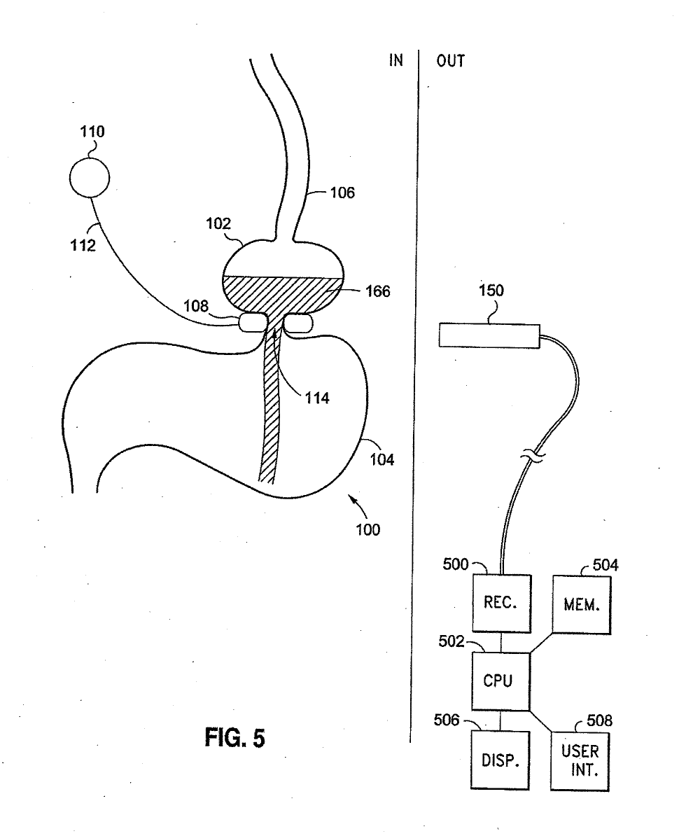

In some embodiments, shown in FIG. 5, there is included a sensor means 150 capable of sound detection that is used to monitor flow of a known volume of a test substance, in this particular case a sound-producing fluid 166 that has been ingested by the patient, past the gastric restriction device 108. The sensor 150 in this case is able to detect sound, and so a suitable sensor can include a microphone, stethoscope, electronic stethoscope or other suitable sound wave sensors known in the art, including for example an ultrasound probe and detector combination. The microphone or other sensor device will be most effective when placed on the patient near, or directed towards, the location of the gastric restriction device, or the flow to be detected, when the patient is in a relatively upright position. As the test substance nears the target area, an increase in sound intensity is detected, which becomes maximal as the fluid flows through the stomal opening 114, or past the target area, and decreases once fluid has passed into the lower portion of the lower stomach pouch 104

In some embodiments, the sound-producing fluid is an effervescent solution comprising effervescent granules taken with water, for example sodium bicarbonate and tartaric acid in water. Other effervescent solutions are also compatible with the present disclosure and so the specific composition is not meant to be limiting. For example, the solution may comprise gas-producing substances such as carbon-dioxide embedded candies as described in U.S. Pat. Nos. 3,012,893; 3,985,709; 3,985,910; 4,001,457; 4,289794, the contents of which are incorporated herein by reference.

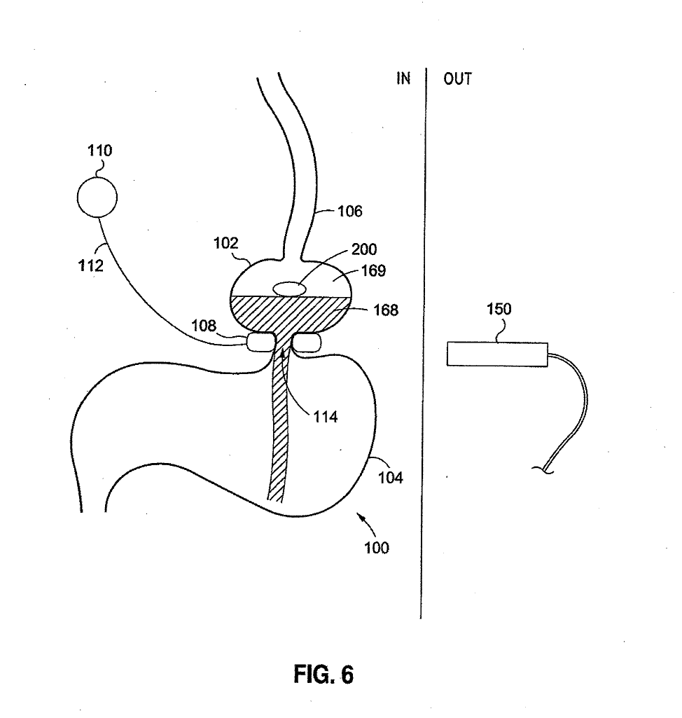

In some embodiments, as illustrated in FIG. 6, the sound-producing fluid is a combination of an ingested substance 168 and a sound-producing capsule 200, such as that disclosed in U.S. Pat. No. 7,160,258 to Imran et al, the contents of which are incorporated herein by reference. The capsule may be biodegradable, or alternatively biocompatible such that is passes safely through the body. The capsule 200 may be free in solution such that it passes through the digestive tract and is eventually expelled, or secured by a line or tether to provide for removal from the patient immediately at the end of a test session. The capsule may be chosen such that its mean density is less than that of the ingested substance 168, so that the capsule floats at the surface of the ingested substance 168, thus marking the interface between the ingested substance 168 and the overlying airspace 169 present in the upper stomach pouch 102. Conveniently the ingested substance 168 may comprise a fluid such as water or any other suitable fluid.

The sound produced by the capsule is in the audible range in some embodiments, and in some embodiments it is ultrasonic or subsonic. Accordingly, the acoustic signature of the capsule 200 may be selected in order to more readily distinguish the sound emitted from the capsule from normal body sounds, such as those occurring in the heart and circulatory system, as a result of breathing, or due to normal peristaltic action or trapped gas in the gastrointestinal tract. Likewise, in some embodiments, during the course of the test, the sound of normal body noises is subtracted from the output signal using an active noise cancellation technology that discriminates between the acoustic output of the capsule and any other noises.

Similar improvement in detection might also be provided using a band pass filter to limit the frequencies detected to those most characteristic of the particular sound-producing fluid being employed. Using these methods either alone or in combination, the signal to noise ratio is increased and the top of the fluid level is sensed while it is in the upper pouch, until it passes through the stoma opening. After passing through the stomal opening, the fluid, and thus the capsule 200, quickly travel to the bottom of the stomach, assuming the patient has followed instructions and not eaten for several hours prior to the test, and sound is no longer sensed at high intensity.

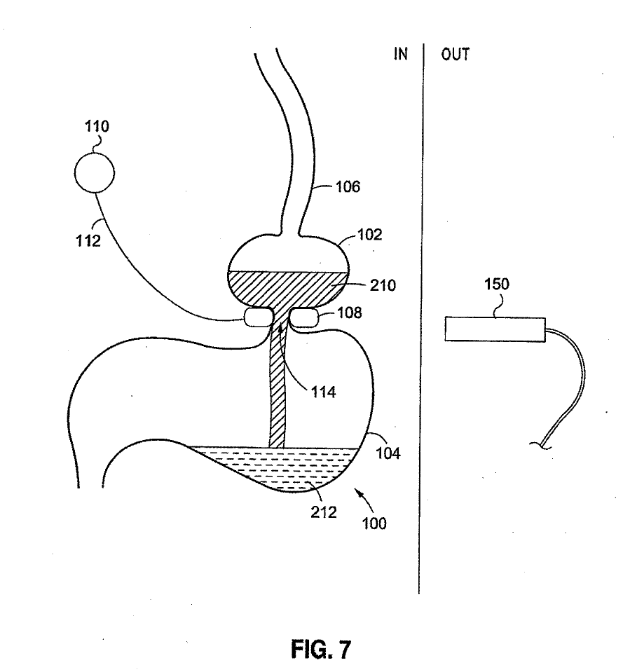

In some embodiments, as in FIG. 7, where an effervescent solution 210 is being monitored, an additional variation in the procedure is added to improve the accuracy of determining when the solution has passed from the upper stomach pouch 102 to the lower stomach pouch 104. In this case a pH-buffered solution 212 is first ingested and allowed to fill a portion of the lower stomach pouch prior to the drinking of the test substance, which in this case comprises an effervescent solution 210. The pH of the buffered solution is selected so that it neutralizes the effervescent solution when the two came into contact. As the effervescent solution passes through the stomal opening 114 into the lower stomach pouch 104, it will come into contact with the pH-buffered solution 212. The mixing of the two solutions in the lower stomach pouch will result in rapidly reduced effervescence, resulting in a similarly rapid decrease in sound levels, in turn leading to more accurate determination of when the contents of the upper stomach pouch have substantially emptied into the lower stomach pouch, due to elimination of significant residual sound.

The disclosure further provides a plurality of test substances of varying viscosity in order to mimic the flow of different types of food or beverage that a patient would normally consume. During a single testing session, preferably the method would be performed at least one additional time, using solutions of differing viscosity, as a means to evaluate restriction device performance for a variety of foods or beverages. The choice of solutions or number of tests performed during a single session is not limiting.

The disclosure further provides a means of warming the substance to be ingested to a pre-determined temperature, such as body temperature, in order to minimize viscosity changes as the test substance warms up after ingestion, or to mimic the normal temperatures of food that the patient would consume. For example food and beverages may be consumed hot or cold, and it is known that viscosity changes with temperature. The choice of temperature for the substance ingested is therefore not limiting to the scope of the invention.

In some embodiments, as illustrated in FIG. 5, the output from the sensor 150 goes to a receiver 500. A processor 502 may also be used for performing the task of timing the beginning and end of the presence of a characteristic sound correlated with flow, and for performing rate flow calculations, and a display 506 for displaying the results of the test to the user. The processor 502 can include, without limitation, a microprocessor. Some embodiments farther include a user interface 508 to enable input of data to the processor 502, or for any other operations that are well known to those skilled in the art, including, but not limited to, inputting patient information, such as recent success or difficulty in losing weight, date and time information, information about the type, volume or temperature of solution ingested, for example. There may also be included a memory portion 504 in order to store data from tests or other relevant information.

By providing amplification, filtering, or other signal processing as appropriate, the sensor 150 can detect noises produced by turbulence, or disturbed flow, that occur when a test substance flows through a gastric lumen, for example, a stomal opening. Thus, in some embodiments, unmodified water in its dynamic state may serve as a sound producing fluid.

### Doppler Ultrasound

In addition to simple detection of sounds produced by an ingested substance, methods of measuring flow rate or residence time, based on Doppler ultrasound, are also contemplated in the present disclosure. For example, a sensor could comprise a Doppler ultrasound probe and detector combination, in order to detect and monitor the movement of the test substance past the gastric restriction device. Testing has demonstrated that a Doppler fetal heart monitor is effective in detecting the passage of fluid moving from the upper stomach pouch to the lower stomach pouch in a patient having a gastric restriction device in place. Therefore, an ultrasound monitoring device intended for clinical use, or one that is suitable for home use, such as a Bistos Hi-Bebe® BT-200, 2 MHz fetal heart monitor or similar device, can be used to detect the presence and movement of fluid from the upper stomach pouch to the lower stomach pouch.

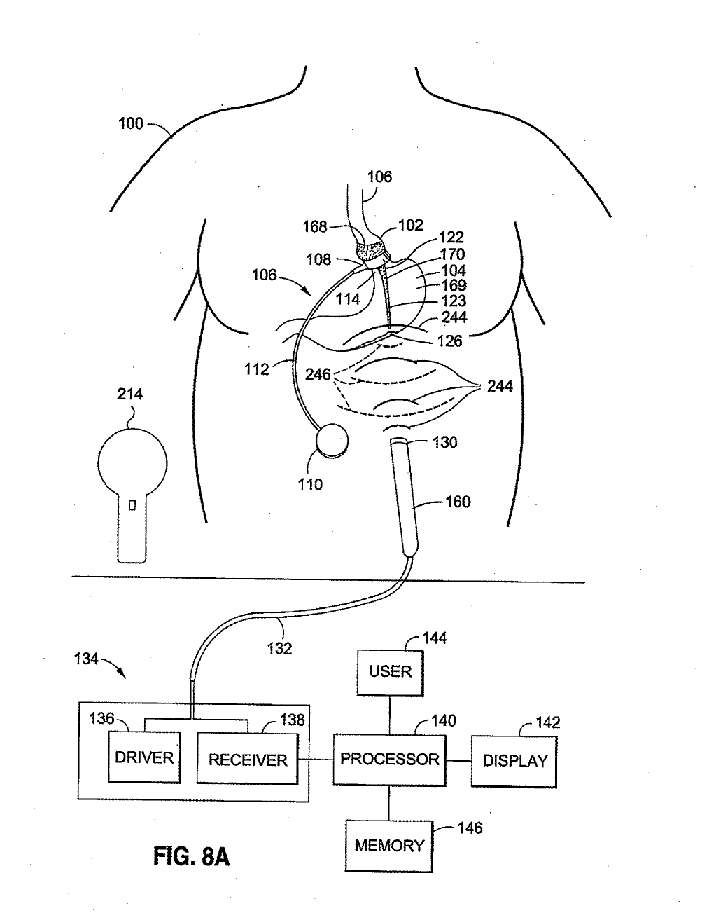

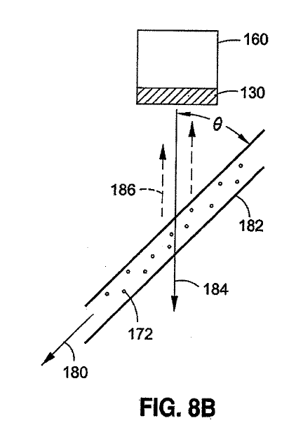

FIG. 8A illustrates an embodiment of an apparatus and method of using Doppler based ultrasound to monitor flow of a test substance in a bariatric patient with a typical gastric restriction device 108 implanted around the stomach, just below the esophagus 106. The gastric restriction device 108 controls the size of a stomal opening 114 between an upper stomach pouch 102 and a lower stomach pouch 104. In some embodiments, the size of the stomal opening is changed by adjustment of an implantable interface 110, operated by an external means 214. The implantable interface 110 transfers the action on the interface to the gastric restriction device 108 via a line 112. Forms of control of the gastric restriction device could include, without limitation, magnetic, inductive coupling, sonically activatable coupling, compressible pressurization members such as diaphragm and valve combinations, ports for injection or withdrawal of fluid, all of which are capable of providing ways in which to open or close the aperture of the restriction device and in turn regulate the stomal opening.

In order to determine whether the stomal opening provided by the aperture of the gastric restriction device 108 is of the desired size (i.e. provides the desired flow rate), some embodiments provide a method for analyzing flow rate of a test substance using non-invasive means that obviates the need for radiographic monitoring procedures. In some method, the patient drinks a known volume of a test substance 168, conveniently comprising a fluid of known volume and viscosity. The test substance 168 fills a portion of the upper pouch 102 and begins to pass through the stomal opening 114, first as a slow moving portion 122 and then, due to the acceleration of gravity, as a faster moving portion 123. A Doppler probe 160 having a transducer 130 is placed against the skin of the abdomen, preferably below the ribs, and relatively near, or below, the location of the restriction device. Ultrasonic gel is optionally placed in the interface between the transducer 130 and the skin for proper acoustic impedance matching. The Doppler probe 160 is oriented so that the transducer 130 sends ultrasonic pulses 244 towards a desired target area, in this case the vicinity of the stomach. Return echoes 246 are received by the same transducer, in between output pulses.

Depending on the acoustic impedance of the material into which the output pulses are directed, the ultrasonic pulses 244 may be reflected as return echoes 246, as in FIG. 8A. Return echoes are created when there is a difference in the acoustic impedance between two regions or materials. For example, a stomach completely filled with pure water produces little echo, as the acoustic impedance of water is very similar to that of skin, fat, muscle and other body tissues. In contrast, there will be a significant difference in acoustic impedance between water contained in the stomach and an air or gas region lying adjacent, as would occur when the stomach is less than completely full.

Medical Doppler systems take advantage of the Doppler effect, in which a Doppler frequency shift (the difference between the original ultrasound pulse frequency and the return frequency) provides information about relative motion. The typical velocities of fluids being probed in medical applications create Doppler shifts with frequencies that lie within the audible spectrum (i.e. 20 Hz-20 kHz). This sound can be calibrated to provide a flow velocity, as is done in cardiac ultrasound applications. In the case of a gastric restriction device, it is not always possible to directly derive flow rate from flow velocity. This occurs primarily because the aperture of the gastric restriction device is not necessarily predictive of the actual size of the stomal opening that it produces in vivo. This occurs due to variability in stomach wall thickness, as well as in the precise location of the restriction device from patient to patient. Testing has shown that the fluid motion through the stomal opening can be detected using a Doppler ultrasound instrument.

Thus, some embodiments, take advantage of the difference in acoustic impedance at the interface 170 between the test substance 168 and the adjacent airspace 169 as a means of “marking” and monitoring the progress of the interface 170 between the two as the substance 168 in the upper stomach pouch 102 moves to the lower stomach pouch 104. Thus, while a simple fluid such as water is relatively poor in terms of providing a media for distinguishable return echoes, echoes are produced as the ultrasound signal encounters the interface between the fluid and the adjacent airspace, and these can be received by the transducer and outputted as a useable signal.

In some embodiments, as shown in FIG. 8A, the Doppler probe 160 is connected to a Doppler control unit 134 via a cable 132. The Doppler control unit will include an ultrasonic driver 136 for producing an ultrasound signal that causes the transducer 130 to oscillate, producing ultrasonic pulses 244. When a pulse is scattered, and an echo is created, the transducer 130 is then caused to oscillate (at a loss of power) by the return echo 246, and the transducer 130 in turn creates a signal that travels to the receiver 138. An ultrasound instrument will typically include a processor 140 and display 142 to manipulate data and provide an output to the user. The control unit may further include a user interface 144 useful in programming the processor 140.

The transducer 130 is preferably configured to vibrate at a frequency in a range of from about 0.5 MHz to 3 MHz. An angle θ is defined as the angle of incidence between the pulses 184 and the direction of fluid flow 180, for example in a tube 182, as illustrated in FIG. 8B. Scattering agents 172 enhance the production of return echoes 186.

If transducer frequency is defined as ft then the Doppler shift frequency (f_{d}) is:

$${f}_{d} = \frac{2 {f}_{t} V cos θ}{c} \qquad Eq . 2$$

where c is the speed of sound in tissue and V is the measured velocity of the fluid or object in motion. Solving for velocity:

$$V = \frac{{f}_{d} c}{2 {f}_{t} cos θ} \qquad Eq . 3$$

With respect to adjusting a gastric restriction device, there are at least two forms of output that will generally be useful. First, detecting a flow condition can be an effective means by which to adjust the gastric restriction device. Determining a flow condition can be as simple as determining whether there is flow, or no flow, past the gastric restriction device. For example, in some embodiments it is desirable to adjust the restriction device so that it is in a substantially closed position, thus providing little or no opening between the upper and lower stomach pouches (i.e. a no flow condition), and then open the device until a flow is just detected. While this is a qualitative adjustment, it corresponds to a fairly aggressive adjustment of the device, and would in turn result in more effective weight control as the amount of food a person could consume comfortably would be quite small.

In contrast, the desired output can be an average flow rate, calculable from the flow duration (i.e. the time from which a volume of test substance begins to flow through the stomal opening to when it has completed flowing through the stomal opening). In some embodiments, an automated timing mechanism starts and stops a timer based on pre-determined threshold values in order to determine a time interval based on detection of the test substance as it flows from the upper stomach pouch to the lower stomach pouch. Knowing this time interval and the volume of the test substance ingested, the following calculation will yield an average flow rate.

Flow rate (mL per second)=Volume (mL)/Time (sec) Eq. 4

This calculation can be done manually by manual timing and manual calculation or by using a computer processor, as in FIG. 8A, for example. Thus, in some embodiments there is included a processor 140, preferably a computer microprocessor, that can be programmed to perform this calculation, and a display means 142 that permits the user to view the results of the flow rate test. There may also be included a user interface 144 that can be used to program the processor, or with which to input any other data relevant to the test session.

The processor 140 may optionally include a memory portion 146 for storing data so that multiple tests with solutions of different viscosities can be made during one testing session and compared, or tests from different sessions can be saved and compared at a later time. The memory portion this provides for storage of data from a plurality of flow rate calculations. Comparison of test runs from different sessions can take into account known diurnal variation in the operation of gastric restriction devices.

Variations in flow rate, or flow condition, that significantly depart from otherwise normal variability can provide an early indication that the restriction device is not functioning properly, has slipped from its implantation site, or needs to be adjusted to maintain an optimal flow rate through the restriction. Storing data from multiple test sessions would also be of use to a physician who is monitoring a patient's status over a period of time. Furthermore, other problems related to the use of gastric restriction devices, such as gastric erosion, might be detected earlier allowing the physician to intervene at a relatively early time to avoid more serious complications. A patient can also have an implanted radio frequency identification device (RFID), which can be read from or written to an optional telemetry unit. The RFID could be used to store a variety of pieces of data including, but not limited to, personal patient information or information regarding adjustment of the gastric restriction device, and a patient's weight, for example.

In some embodiments, the display 142 may provide an audible, visible, or tactile indicator to direct the user to start or stop a manual timing device, or to indicate a flow or no flow condition, thus letting the user know when stop adjusting the device. The alert might be as a simple as an audible tone, a flashing light or LED, a device that vibrates, or a heat source. Other types of alerts could include, without limitation, video displays and other types of displays well known in the art. In more sophisticated embodiments, the display may provide a readout from the computer processor of the result of a flow rate calculation, providing a calculation in mL per second or some other convenient measure.

The computer processor and display may also provide additional functionality such as being able to program in the volume and viscosity of the test substance, or volume and temperature information. Even more elaborate data processing may include a programmable correction function to account for situations where the test substance is at a temperature other than body temperature in order to provide a corrected flow rate.

Where the flow rate measurement is conducted using water as the test substance, optimal detection will be achieved as long as the Doppler probe 160 is pointed generally towards the interface 170 between the water and stomach gas, as this interface creates echoes as a result of acoustic impedance differences. Where a flow condition is being determined (i.e. flow versus no-flow), the target area may include a portion of the interface near the stomal opening, or a location at a distance below the stomal opening.

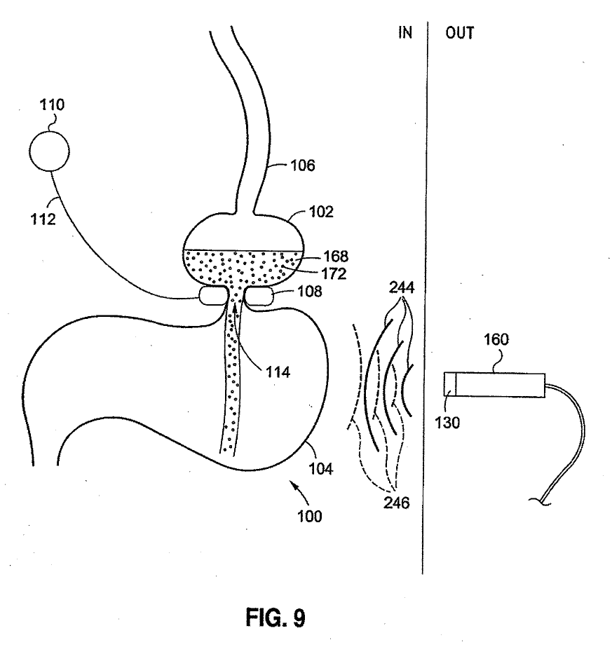

In some embodiments, as illustrated in FIG. 9, the test substance 168, preferably a fluid, may include a scattering agent 172 that serves to scatter ultrasound waves 244 and enhance the creation of return echoes 246. Scattering agents suitable for use with ultrasound systems are well known in the art and may include such things as flax seed, micro-bubbles or micro-spheres, microscopic ingestible kaolin particles, such as those described in U.S. Pat. No. 5,179,955 to Levene et al., the contents of which are incorporated herein by reference, or even orange pulp suspended in water can be used.

The use of these scattering agents within the test fluid provides an acoustic impedance difference in the test fluid itself as compared to surrounding tissue, instead of only at the fluid/gas interface in the stomach. Further, barium suspensions typically used in radiographic methods such as the barium swallow method also serve to scatter sound waves and enhance the signal perceived by the Doppler device, and so may be used as a scattering agent within the scope of the present disclosure to increase the production of Doppler shift echoes. For example, a low concentration Barosperse® suspension can be used.

Some embodiments further include a timing means that is activated when the desired sound is sensed above a pre-determined threshold level. Likewise, the timer may be stopped when the desired sound drops below the threshold intensity. Combining time measurements and the volume of material ingested an accurate calculation of flow past the restriction device can be determined. The timing mechanism may further be under the control of a processor such as that described below. In some embodiments the output from the Doppler ultrasound may be saved as a computer file using a sound analysis software program and the data analyzed at some point in the future.

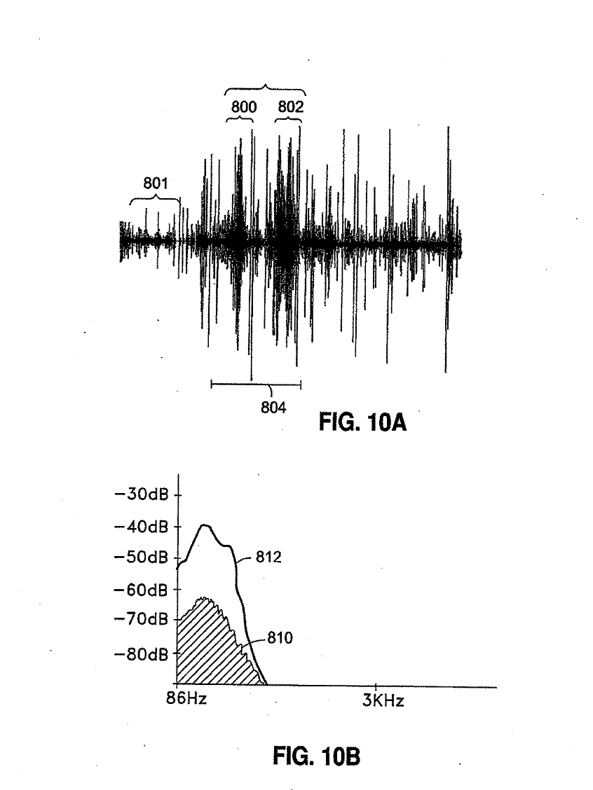

An example of a sonogram from a Doppler ultrasound experiment is shown in FIG. 10A. Movement of fluid through the stomal opening can occur in a pulsatile fashion due to normal gastric peristalsis. As shown, two periods of increased sound intensity 800 and 802 were observed. By comparison, background sounds 801 not related to movement of fluid through the stoma opening are detected but at appreciably lower levels. Barium fluoroscopy performed concomitantly confirmed movement of fluid from the upper stomach pouch to the lower stomach pouch during this time.

From this, a time interval 804 can be calculated corresponding to the time it takes all the material in the upper stomach pouch to move through the stomal opening into the lower stomach pouch. Spectral analysis of baseline 810 and fluid movement-based 812 Doppler echo returns as in FIG. 10B shows during movement of fluid through the stomal opening, not only does intensity of Doppler return echoes increase, but that return signals have distinguishable spectral characteristics.

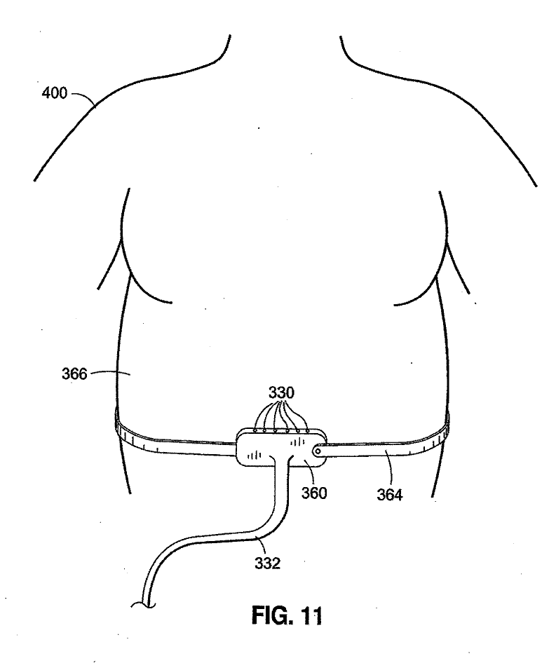

In some embodiments, as illustrated in FIG. 11, the Doppler probe 360 is a linear array probe having a relatively wide contact surface. The array includes a plurality of transducer elements 330. A strap 364 is attached to the Doppler probe 360 for securement around the torso 366 of the patient 400. When the Doppler probe 360 is secured by the strap 364, the operator is now free to use both hands on equipment related to the adjustment of the gastric restriction device. The wide array of the probe 360 allows for improved ability to correctly aim the transducer elements 330 at the target area. In some embodiments, the signals to and from the control unit (not shown) travel via a cable 332. In some embodiments, signals to and from the control unit may be transmitted via a wireless connection.

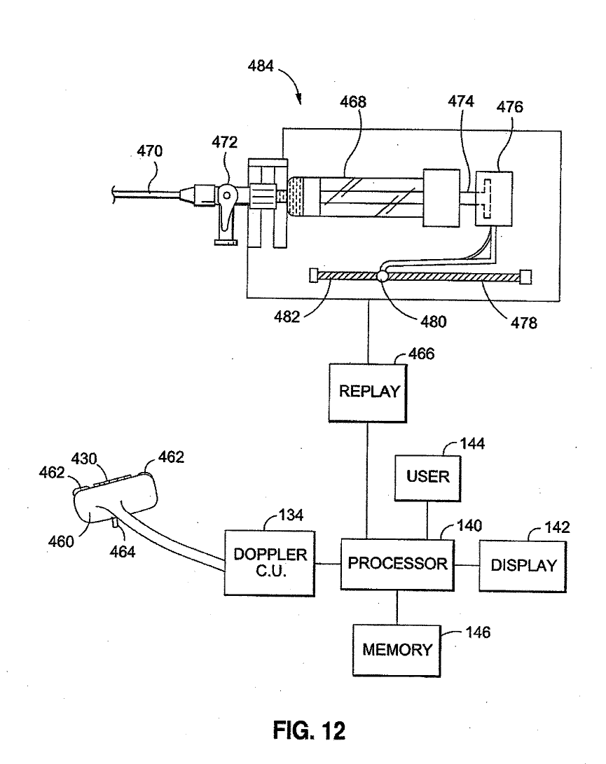

In some embodiments, such as that illustrated in FIG. 12, there may be provided other methods of securing the Doppler probe 460 to the patient to permit hands-free operation. For example, the Doppler probe 460 may be secured using adhesive strips 462 commonly use in medical applications. In addition, the Doppler probe 460 might provide a port 464, or access, to allow injection of gel into the contact area between the patient and the probe in order to improve acoustic coupling between the transducer elements 430 and the skin. Other means for securing the probe to the patient in order to permit hands-free operation are also contemplated and will be readily recognized by those skilled in the art. As a result, the means by which the probe is secured or placed on the patient is not a limiting feature of this disclosure.

FIG. 12 further illustrates an embodiment for automatically adjusting the size of the stomal opening. In this embodiment, a system with a hydraulically adjustable gastric restriction device is shown, but it is also contemplated that other types of devices could be controlled in this way such as, without limitation, those adjusted by magnetic drive, inductive coupling, and any other remotely or direct drive systems operative to adjust a gastric restriction device. A needle 470 is placed subcutaneously through the injection port of the gastric restriction device (not shown). A valve 472 is in open position, and a saline-filled syringe 468 which is part of an aspiration/injection system 484 is attached to the needle 470 and saline is injected until the gastric restriction device fully constricts the stoma. In one embodiment, a syringe plunger 474 of a syringe 468 is connected to a drive, which in the illustrated embodiment is a screw 482 and nut 480 combination, coupled to a syringe plunger holder 476 that engages the syringe plunger 474. Other means for driving the syringe plunger 474 in and inwards or outwards motion are also possible and will be readily known to those skilled in the art.

To begin, the patient ingests the test fluid and the Doppler ultrasound instrument is started with a pushbutton, or through the user interface 144, such that it begins producing ultrasonic pulses and detecting Doppler shift echoes, thus allowing monitoring of flow through the stomal opening. The valve 472 is placed in the open position, and the gastric restriction device is inflated by injection of saline from the syringe 468 through the injection port into the gastric restriction device. Injection of saline may be done manually, or the relay 466 may signal a drive to turn the screw 482 and nut 480 combination such that the syringe plunger 474 is moved into the syringe 468, injecting saline into the restriction device.

As the restriction device is filled with saline, the stomal opening becomes more restricted. Once the restriction device is sufficiently inflated, the stomal opening is occluded and no flow occurs. At this point, the Doppler will not sense any return echoes, consistent with the no-flow condition. Conveniently, an audible, visual, or tactile alarm or other type of suitable alert can be provided to indicate that a no-flow condition has been achieved. Alternatively, the relay 466 can automatically stop movement of the drive so that no more saline is injected. After a no-flow condition is confirmed, the relay will start the syringe drive in the opposite direction, such that the syringe plunger 474 will be withdrawn from the syringe 468, thus removing saline from the restriction device. As the restriction device is “deflated” the stomal opening opens, and flow from the upper stomach pouch to the lower stomach pound occurs. When Doppler shift echoes are sensed at a level above a pre-determined threshold, indicating a desired flow condition, the processor 140 will communicate to the relay 466 and stop the evacuation of the syringe 468. The valve 472 is then closed to maintain the hydraulic gastric restriction device at the appropriate adjustment setting. The valve 472 may also be used to add saline to the syringe 469.

An object of the present disclosure is to provide an accurate measure of flow rate through the stomal opening produced by a gastric restriction device. However, depending on the nature of the material being consumed (e.g. fluid or food) flow rate may vary. For water, the desired flow rate ranges from about 1 mL to about 20 mL second. In contrast, a slightly more viscous solution such as a dilute BaSo4 suspension in water may have a slower flow rate depending on the amount of barium included in the suspension. Much more concentrated BaSo4 suspensions are commercially available, for example E-Z-Paque®, and have viscosities many times greater than water over the typical flow rates encountered in clinical applications. Solutions with even higher viscosity will be expected to move even more slowly through the opening. For example, it is known that solid food may be blocked by a stomal opening where liquids like water will readily pass. Therefore, another object of the disclosure is to provide a means of measuring flow rates with solutions having varying viscosity in order to better model the behavior of the various foods or beverages that the patient might normally consume, and thus derive an optimal flow rate.

This may be accomplished through the use of test substances of varying viscosity in order to mimic the flow rate of a variety of ingested materials. For example water at 20° C. has a viscosity of about 1 cP. Solutions with varying amounts of sucrose present can have viscosities ranging from about 3 cP to about 3,000 cP. Vegetable juices can have viscosity values ranging from less than about 10 cP to greater than about 3,000 cP. Solid foods have even higher viscosity values, as high as about 1×105 cP or even greater. Thus a low viscosity test substance might be one with a viscosity of less than about 10 cP, a medium viscosity test substance might be in the range from about 10 cP to about 10,000 cP, and a high viscosity substance might have a viscosity from about 10,000 cP and higher. In some embodiments a fluid having a viscosity in the range of about 0.5 to about 2 cP can be used.

Thus, in terms of usefulness of the data obtained in testing flow condition or flow rates, it will be desirable within a test session to determine either flow condition or flow rates for substances of differing viscosity. Thus, it is possible to not only to check for flow through the stomal opening, but to ensure that the opening can accommodate desired rates of flow over a range of substance viscosities typical of fluids and foods ingested by most people. For greater certainty regarding the function of the restriction device, low, medium and high viscosity test fluids may be tested in turn as part of a single testing session, and in this way the most beneficial adjustment of the gastric restriction device may be made based on an optimal flow condition or flow rate. As the test is relatively easy, non-invasive and of relatively short duration, testing multiple fluids would not be particularly burdensome to the patient, and would potentially provide the physician or other caretaker with the best possible information as regards the functioning of the gastric restriction device in order to adjust the device to provide an optimal flow rate or flow condition.

Water is a preferable test fluid, especially when testing highly constricted stomal openings, as water has a relatively low viscosity and thus will flow relatively unimpeded through a wide range of stomal opening sizes. Viscosity is also affected by the temperature of the material, such that as temperature increases viscosity typically decreases. For example, water has a viscosity of about 1 cP at 20° C., which decreases to about 0.69 at 37° C. Thus, it would be advantageous to provide a means of equilibrating the test fluid to a pre-determined value prior to ingesting in order to reduce test to test variability. For example, the test fluid could always be heated to a temperature close to body temperature (37° C.) in order to minimize changes in fluid viscosity that would occur as the fluid warms in the body upon ingestion.

### In vitro Flow Measurements

In vitro flow experiments were conducted in order to evaluate the relationship between restriction diameter, solution viscosity, and flow rate. To evaluate viscosity effects, four different solutions were used at room temperature: water; Barosperse® water (2:1 by volume); Barosperse®: water (1:2 by volume); and “simulated” Gastrografin® (67.5% glycerin, by volume, in water). To test flow rate, these solutions were allowed to flow through a vertically oriented tube, occluded with a plug having a lumen of defined size functioning as a flow restrictor. The lumen through the plug simulates a stomal opening as would be produced by a gastric restriction device. Several different plugs were used, with lumen diameters ranging from 4-12 mm. For each experiment 50 mL was applied to a funnel atop the tube, and the time taken for substantially the entire 50 mL to pass through the “restriction” (i.e. the lumen of the flow restricting plug) was determined.

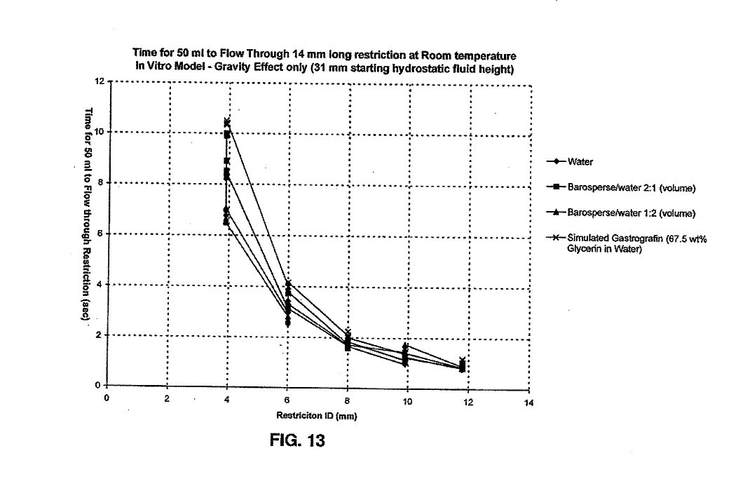

As shown in FIG. 13, as the diameter of the restriction (i.e. the diameter of the plug lumen) in increased, the time for the 50 mL to flow through past the simulated restriction decreased. At smaller restriction, for example at 4 mm, viscosity also affected flow rate such that the more viscous Barosperse® water (2:1) and simulated Gastrografin® took significantly longer than water to flow through the restriction.

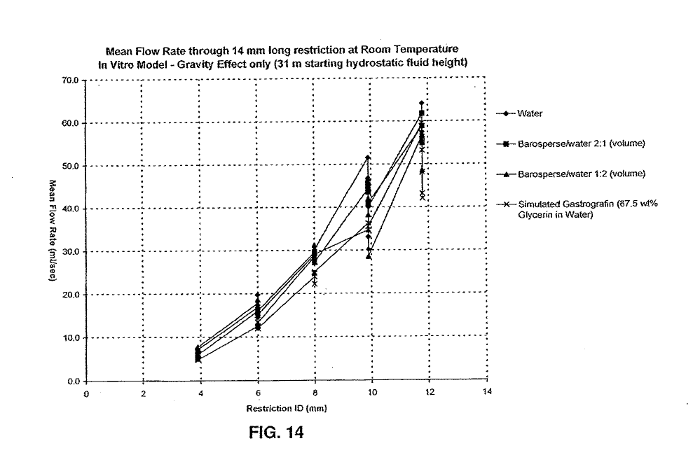

FIG. 14 shows that as restriction diameter increases flow rate also increases, such that a 3-fold increase in restriction diameter, results in an approximately 6-fold increase in flow rate. As desired flow rates are typically in the range of about 5 mL to about 15 mL per second, these results would suggest that in practice, very small stomal openings are going to be desired.

As an object of the disclosure is to provide an accurate, yet non-invasive, method of measuring flow rate, or flow condition, past a gastric restriction device, it will be of particular advantage to provide a test in which variability of various test parameters is minimized. As discussed above, the volume, temperature and viscosity of the test substance are among the factors that will affect the data recovered from a flow rate test as practiced by embodiments of the present disclosure. In order to minimize variability inherent to the test method, and maximize the accuracy of the test results, some embodiments provide a kit with test substances comprising standardized test solutions, instructions on how to perform the test to achieve maximal accuracy and reproducibility, and optionally a Doppler ultrasound instrument for suitable for home or clinical use.

The kit may include a set of standard test solutions of pre-determined viscosity, for example a low viscosity, medium viscosity and high viscosity solution to evaluate flow of different types of materials through the stomal opening. For further ease of use the test fluids could be pre-packaged in a one-use form of a known volume of fluid. By using a pre-packaged solution, the patient would use the correct volume of solution without incurring a risk of measuring error. As it might be further advantageous to ingest different volumes of fluids depending on their viscosity in order to obtain the most accurate measure of flow rate, pre-packaging test fluids in kit form would provide a simple way in which to provide test fluids of varying viscosities, that are also optimized for volume. The kit could further include a heating device to heat the solution packages to a pre-determined value, for example 37° C., generally accepted normal human body temperature to minimize any changes in viscosity that would occur upon ingesting a test solution. In some embodiments the kits may further provides solutions of different viscosities for use at different times of the day. It is known that flow past gastric restrictions exhibit diurnal variation, and so ingesting a solution with a higher viscosity when testing later in the day may be more useful.

The test solutions could be further coded with a simple letter or number code (e.g. A, B, C or 1, 2, 3) and the coding could be used in conjunction with a calibration system on the Doppler instrument such that a correspondence algorithm would reference the solution code as pertaining to a particular volume and viscosity previously programmed or programmable into the processor. Coding would also minimize operator errors in terms of inputting volume or viscosity measures, values which would typically comprise multiple digits, and whose input could be prone to operator error.

In some embodiments, the kit further includes a Doppler ultrasound instrument system suitable for home or clinical use. The system may include additional automated features whereby the instrument is calibrated by input of the solution codes as described above. A patient or their caretaker can be readily trained on the setup of the instrument including the input of test fluid codes, as well as the operation and correct placement of the Doppler probe. A patient may setup the instrument, ingest a test fluid and swallow the test fluid while operating the Doppler probe, and the instrument would make the appropriate measurements based on echoes received, and calculate a flow rate, or a simple flow condition evaluation could be performed. Having done this, a patient could then relay the results of the test to their bariatric physician, who could decide whether, based on the flow test, adjustment of the device would be indicated.

Optionally, someone other than the patient could perform the monitoring steps. Flow rate information can then be provided to a physician or other person qualified to adjust the restriction device in order to make adjustments of the restriction device to provide an optimal flow rate. Departure from normal flow rates could also inform a patient that a visit to a physician to evaluate the operation of the device is in order, or may signal the initial stages of other problems that may require medical attention, such as device slippage or gastric erosion. A telemetrically adjustable band could conceivably then be adjusted over the telephone.

As explained in the examples provided, the disclosed system allows for a diagnostic procedure to quantify and adjust the stomal opening produced by a gastric restriction device, reducing or eliminating the need for radiation from X-ray fluoroscopy, or other invasive procedures. Minimizing exposure to ionizing radiation in the form of x-rays is an advantage for any patient, but in particular it provides a special advantage in the context of bariatric procedures, as many bariatric patients are females of child-bearing age who may be pregnant without being aware, and thus should not be unnecessarily exposed to radiation. There is also an economic advantage to avoiding radiography as fluoroscopy is a relatively costly procedure, and the overall cost is exacerbated if there is a need to continually monitor the gastric restriction device over an extended time as might be possible in long-term monitoring of a restriction device. There is a further advantage in that testing can be done at home. This permits greater ease in testing, likely improves patient compliance, and allows for testing at various times of day to account for normal diurnal variation in the functioning of the restriction device. Home testing also avoids the need for timely and costly visits to a clinical setting.

Using any of the embodiments described above or their equivalents, data collection could be easily performed by a patient or their caretaker. Further, the data may be displayed as either an audible or graphic output in real time, or saved as an electronic file for later evaluation by a person qualified to interpret the data collected, for example a physician. A further advantage would be realized by combining the sound detection system, or Doppler ultrasound instrument, with a recording interface and a commercially available software package to allows storage of sounds in various formats, for example as “.wav” format sound files. The recorded data could then be forwarded physically or electronically to a physician for subsequent evaluation.

As these files are easily created and stored, a number of tests could be performed with the advantage that data from different points in time could be collected and analyzed at some future date for comparative purposes. Comparison studies would make it easy to establish standardized criteria with which to calculate flow rates, or to detect changes in the functioning of the gastric restriction device over time. By comparing flow rate with weight loss, a physician could carefully monitor a patient's progress in order to maximize the efficacy and safety of a bariatric program.

In addition to the increase in reliability of the adjustment procedure related with the teachings of the inventive material, patients have a more positive sense that a significant improvement has been made to their status, in association with a dedicated piece of equipment having a validated function. This further aids the patient's progress, as there is an additional psychological motivation, very important in most weight loss situations.

Some other methods attempt to use a patient's ability to sense movement of water through the stomal opening as an indicator for adjusting the device. However, a patient's ability to sense the passage of water is typically inconsistent, especially from patient to patient. Some patients are better at sensing when water passes than others, even when aided by the use of cold or hot water. As a result, is difficult for the physician to adjust a device based on patient feedback. In addition, even in those patients who are able to sense fluid movement, this ability can be reduced over time for a variety of reasons, including a dilated esophagus, or other esophageal anomalies. In some cases, these esophageal conditions may even cause the lower portion of the esophagus to act more like an extension of the upper pouch of the stomach.

Instead of the Doppler sensor, if a test fluid comprising barium or other metals in water is used, an external metal detector can be used analogously to determine when the test fluid is flowing.

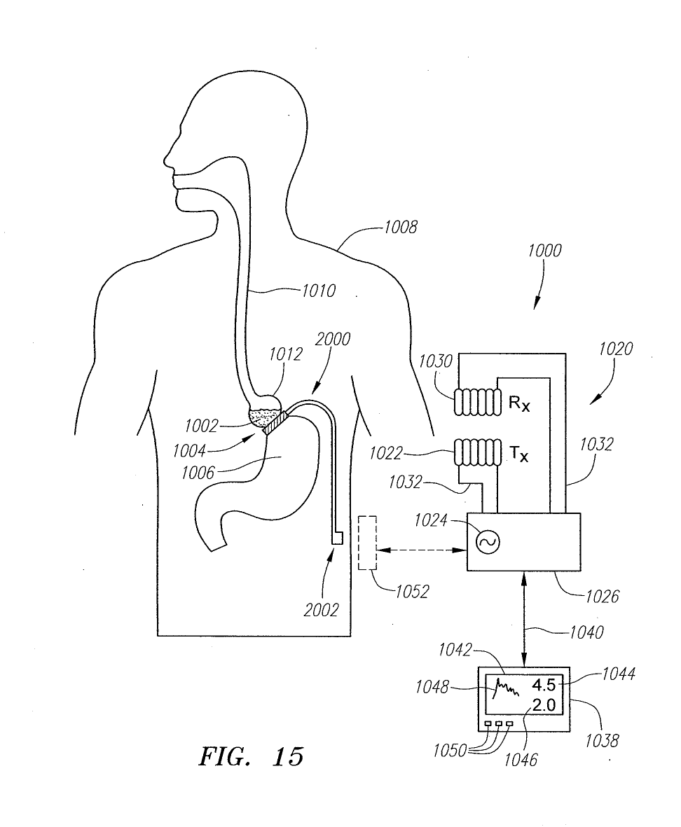

FIG. 15 illustrates a system 1000 for determining the flow rate of fluid 1002 passing through a restricted portion 1004 of a gastric lumen 1006 of a patient 1008 according to another embodiment of the invention. In this embodiment, the fluid 1002 is magnetically detectable fluid. That is to say, the fluid 1002 is capable of being sensed by an externally located magnetic sensor 1020 (described in further detail herein). The fluid 1002 may include magnetically permeable or magnetically susceptible fluids. For instance, the fluid 1002 may be ferromagnetic, paramagnetic, superparamagnetic, diamagnetic, or conductive. Alternatively, the fluid 1002 may include a carrier fluid that contains particulates with ferromagnetic, paramagnetic, superparamagnetic, diamagnetic, or conductive properties. For example, the fluid 1002 may contain a metal or metallic species that has properties that allow it to be detected by the magnetic sensor 1020.

The carrier fluid is preferably a biocompatible fluid such as water or oil. The magnetic component may include a plurality of particles or other particulate matter. One illustrative example of a magnetic component includes particles of magnetite (Fe₃O₄). Another example includes gadolinium compounds. The particles may have sizes on the order of micrometers or even nanometers. Optionally, the fluid 1002 may contain a surfactant that enhances the overall mixing between the magnetic component and the carrier fluid to form a well-mixed suspension. Alternatively, the magnetic particles may be coated with a material such as silicone to aid in forming the suspension. For example, an aqueous suspension of silicone-coated, superparamagnetic iron oxide may be one fluid 1002. One example of such a fluid 1002 is sold under the brand GastroMARK® although higher concentrations of iron oxide are likely needed. The fluid 1002 may also include so-called ferrofluids that have magnetite suspended in either a liquid solvent or oil. These ferrofluids 1002 generally contain about 5% to 10% (by volume) magnetite. Magnetite may also be suspended in an organic carrier fluid. For example, magnetite particles may be suspended in oleic acid.

As stated above, instead of a magnetic component, the fluid 1002 may contain a metallic component. The metallic component may be formed from a plurality of particles or particulate matter and may require the use of a surfactant to aid in forming a well-mixed suspension. Alternatively, the metallic particles or particulate matter may be coated with, for example, silicone to aid in forming the suspension. In another aspect, the fluid 1002 may be formed from an elemental metal. For example, gallium is a liquid at room temperature that is highly conductive. It should be noted that the particles do not necessarily need to be metallic, as any conductive material has the possibility of being sensed. Alternatively, a non-particulate containing ionic solution can be used and sensed by the magnetic sensor 1020.

FIG. 15 illustrates a restricted portion 1004 formed in the patient's gastric lumen 1006. In the illustrated embodiment, the restricted portion 1004 is artificially created through the use a gastric restriction device 2000 positioned about the patient's stomach 1006 (e.g., gastric lumen). The gastric restriction device 2000 generally includes an adjustable band that at least partially or fully wraps around a portion of the patient's stomach 1006 or esophagus 1010 and is connected to an implantable interface 2002 that is located subcutaneously (or elsewhere) inside the patient 1008. In certain gastric restriction devices 2000, the implantable interface 2002 is a port or the like through which the hypodermic needle of a syringe is placed to selectively fill or evacuate fluid to decrease or increase the size of the restriction (e.g., stoma) formed in the stomach 1006. In still other embodiments, the gastric restriction device 2000 may be adjusted in a non-invasive manner, for example, through the use of magnetically-driven implantable interface 2002 that is configured to selectively increase or decrease the size of the stoma via an externally located adjustment device that is operated by the user (or automatically controlled).

While FIG. 15 illustrates the restricted portion 1004 formed in the stomach it should be understood that the system 1000 may be used to determine flow rates of fluid 1002 through other gastric lumens 1006 beyond the stomach. Such restricted portions 1004 may be artificially created or they may be naturally occurring restrictions that are formed due to anatomical abnormalities or even pathological states. In FIG. 15, the esophagus 1010 is shown emptying into the upper or superior portion of the stomach 1006. FIG. 15 also illustrates a bulge or pouch 1012 that is formed in the stomach 1006 upstream with respect to the restricted portion 1004. By restricting the stomach 1006 using the gastric restriction device 2000, the volume of the stomach that is available to immediately receive consumed food and liquid is significantly reduced.

As stated herein, there is a need to accurately determine the flow rate at which fluid passes from the bulge 1012 and into the larger portion of the stomach 1006 located downstream from the restricted portion 1004. The flow rate of liquid passing through the restricted portion 1004 can be used by the physician or other skilled technician to adjust the size of the stoma to properly regulate the flow of food and fluid from the artificially created bulge 1012 in the stomach 1006. FIG. 15 illustrates a system 1000 that is capable of determining the flow rate of the fluid 1002 that passes through the restricted portion 1004. The system 1000 accomplishes this by determining the quantity of the fluid 1002 contained in the bulge 1012 at various times (e.g., time intervals). Unless the restricted portion 1004 is fully closed, fluid 1002 will generally pass from the bulge 1012 and into the larger volume of the stomach 1006 via the artificially created stoma. By knowing the volume of fluid 1002 within the bulge 1012 at two time intervals, the flow rate can then be calculated based on the change in volume over time. In one preferred aspect of the invention, the volume of the fluid 1002 within the bulge 1012 is rapidly sampled (e.g., at least once every 0.5 seconds) to give a real time measurement of the flow rate of fluid 1002 passing through the restricted portion 1004.

In certain embodiments, it is possible to use the system 1000 to determine the quantity of fluid 1002 that remains within a gastric lumen 1006. For example, in some patients 1008 that consume too much food despite the placement of the gastric restriction device 2000 may remodel or reform the esophagus which creates pockets or pouches that can trap food and fluid. In these patients 1008, the system 1000 may be able to determine the volume of residual fluid 1002 that remains in these spaces. Likewise, certain patients 1008 have stomachs 1006 that do not fully empty. The system 1000 may be employed to determine the quantity of residual fluid 1002 that remains in the stomach 1006. In addition, the system 1000 may be employed to determine the extent of remodeling of the esophagus 1010.

Still referring to FIG. 15, an externally located magnetic sensor 1020 is provided that is configured to detect a quantity of fluid 1002 contained in the gastric lumen 1006. In the embodiment of FIG. 15, the magnetic sensor 1020 detects the quantity of fluid 1002 contained upstream of the restricted portion 1004 of the gastric lumen 1006. In this regard, the magnetic sensor 1020 detects the quantity of fluid 1002 contained in the bulge 1012 of the stomach 1006 formed by the gastric restriction device 2000. When referring to a quantity of fluid 1002 this may be expressed in terms of a volume or a mass (or both).

The magnetic sensor 1020 includes at least one transmission coil (Tₓ) 1022 that is connected to a source of alternating current 1024. In FIG. 15, the source of alternating current 1024 may, optionally, be integrated into a controller 1026 that houses the circuitry for driving the transmission coil (Tₓ) 1022 as well as contains the sensing circuitry that receives the signal from the at least one receive coil (Rₓ) 1030. The at least one transmission coil 1022 and the at least one receive coil 1030 may have any number of geometries and configurations. For example, the transmission and receive coils 1022, 1030 may be shaped as a polygon, round, oval, non-round, spiral, and the like. The transmission and receive coils 1022, 1030 may be formed from a single conductor or wire or multiple wires, for example multi-strand or multi-filar. One or more of the multi-strand or multi-filar conductors or wires may be twisted. For example, all of the wires may be twisted in a general helical pattern. Of course, the source of alternating current 1024 may be separate from the controller 1026. Preferably, the magnetic sensor 1020 can operate on standard 110 VAC, 60 Hz outlets but this may vary depending on the particular standard used (e.g., European standard VAC lines). The at least one transmission coil 1022 and the at least one receive coil 1030 are connected to the controller via signal lines 1032 (e.g., conductive wires or the like).

The at least one transmission coil 1022 induces magnetic fields through the body of the patient 1008. The at least one receive coil 1030 measures the resultant change in the magnetic fields. This change is generally proportional to the volume change of the fluid 1002. In one aspect, the magnetic sensor 1020 works similarly to a conventional metal detector. The applied magnetic field will induce eddy currents within the fluid 1002, for example, a conductive fluid or ferromagnetic fluid. These eddy currents, in turn, generate a magnetic field that is then sensed by the magnetic sensor 1020. If the fluid 1002 contains a magnetically susceptible material, the presence of the fluid 1002 will change the field strength. This change in field strength can then be detected by the magnetic sensor 1020. It may be desired in some instances to shield the at least one receive coil 1030 from the at least one transmission coil 1022, as is commonly done in commercial metal detectors.

Still referring to FIG. 15, the magnetic sensor 1020 may include a display 1038. The display 1038 may be integrated into the controller 1026 or it may be a separate component, as illustrated in FIG. 15, that is connected via signal line 1040. The display 1038 includes a screen 1042 on which various data may be displayed during use of the magnetic sensor 1020. For example, the screen 1042 may display one or more parameters or indicia that aid the physician or other skilled technician in evaluating the flow rate of fluid 1002 through the restricted portion 1004 of the gastric lumen 1006. For example, the screen 1042 may display the flow rate 1044 of the fluid 1002 flowing through the restricted portion 1004. The displayed flow rate 1044 may be a real time number or it may be an average or median over all or a portion of the test procedure. For example, as shown in FIG. 15, a flow rate of 4.5 ml/second is displayed on the screen 1042. The screen 1042 may also display other clinical meaningful data. For example, the screen 1042 may display the quantity of fluid remaining in the pouch or bulge 1012 above the restricted portion 1004 (e.g., volume or mass), the elapsed time, a target flow rate, or the current size (or configuration) of the gastric restriction device 2000. FIG. 15 illustrates a “2.0” displayed on the screen 1042 which may indicate that the size of the gastric restriction device is currently set at a 2.0 cm setting.

The screen 1042 may also include a trace 1048 of one or more variables over a period of time. For example, the trace 1048 may illustrate the quantity of fluid 1002 passing through the restricted portion 1004 as a function of time (e.g., seconds or minutes). Alternatively, the trace 1048 may illustrate the quantity of fluid 1002 contained in the bulge 1012 upstream of the restricted portion 1004 as a function of time. The display 1038 may optionally include one or more input devices 1050 such as buttons, dials, or slides that can be used to toggle between different modes or views of the display 1038. The input devices 1050 may also be used to adjust the parameters of the controller 1026. For example, the input devices 1050 may adjust the power delivered to the transmission coil 1022 or the gain used to detect the signal in the receive coil 1030. Of course, the input devices 1050 may be used to adjust other settings as well. While the display 1038 has largely been described as using one or more visual images located on a screen 1042 it should be understood that the display 1038 may include audile or even tactile signals that represent an indication of the detected or measured flow rate. For example, the measured flow rate may be an audible signal that changes, pitch, frequency, or amplitude in response to changes in the flow rate. Alternatively, the system 1000 may emit an electronically generated communication (e.g., voice) that can be used to inform the physician or other technician of one or more measured parameters.

Still referring to FIG. 15, in one embodiment, the controller 1026 is operatively coupled to an externally located adjustment device 1052. The external adjustment device 1052 enables adjustment of the gastric restriction device 2000 in a non-invasive manner. For example, the external adjustment device 1052 may include one or more moveable magnetic elements that, when moved, effectuate movement of one or more internally-located magnetic elements located within the implantable interface 2002. Movement of the internally-located magnetic elements can then adjust the size or configuration of the gastric restriction device 2000 to increase or decrease the size of the stoma formed around the gastric lumen 1006. For example, U.S. patent application Ser. No. 11/760,482, which is incorporated by reference as if set forth fully herein, describes various embodiments of non-invasively adjusting a gastric restriction device 2000. In one aspect of the invention, the controller 1026 works in conjunction with the adjustment device 1052 to automatically adjust the size of the stoma in the restricted portion 1004 by making adjustments of the gastric restriction device 2000. For example, the user may program a target or desired flow rate into the controller 1026 (or via display 1038). After the patient consumes the fluid 1002, the controller 1026 would communicate with the adjustment device 1052 to make the necessary adjustments to bring the actual or measured flow rate to the target flow rate.

Of course, the magnetic sensor 1020 may be independent of the adjustment device 1052. For example, the physician may manually adjust the gastric restriction device 2000 using the adjustment device 1052 while watching the readout(s) on the display 1038. Adjustments are made as needed to the gastric restriction device 2000 using the adjustment device 1052 until the target flow rate is reached. It should also be understood that the magnetic sensor 1020 may be used in connection with other gastric restriction devices 2000 that are adjusted using a variety of methods. For example, certain gastric restriction devices 2000 are adjusted by inductive coupling using an external source. Still other gastric restriction devices 2000 may be adjusted by inserting or withdrawing a fluid into the implantable interface 2002 using a syringe or other similar tool.

While FIG. 15 illustrates the at least one transmission coil 1022 being located on the same side of the patient 1008 as the at least one receive coil 1030 it should be understood that the location of the various transmission and receive coils 1022, 1030 may be arranged in a number of geometries or orientations. For example, a transmission coil 1022 may be placed on one side of the patient 1008 while a receive coil 1030 may be placed on an opposing side of the patient 1008.

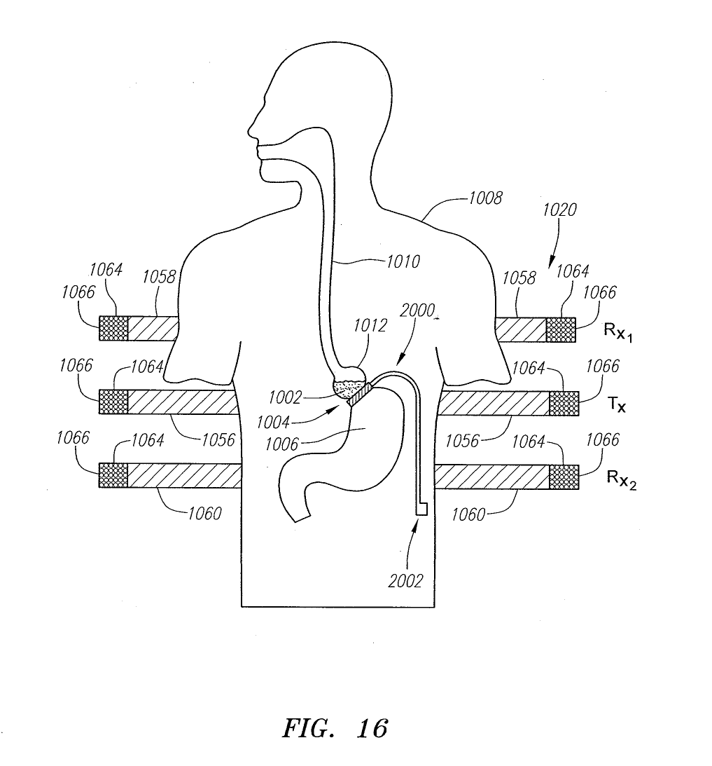

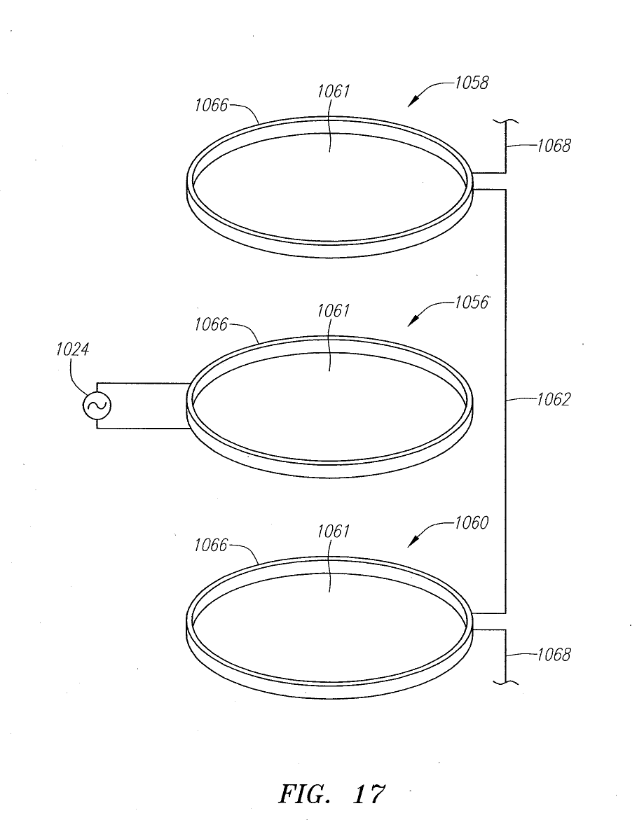

FIGS. 16 and 17 illustrate another embodiment of an externally located magnetic sensor 1020. In this embodiment, the magnetic sensor 1020 includes a transmission coil (Tₓ) 1056, a first receive coil (Rₓ₁) 1058, and a second receive coil (Rₓ₂) 1060. As seen in FIGS. 16 and 17, the transmission coil 1056 is interposed between two outer receive coils 1058, 1060. The amount or degree of separation between each coil 1056, 1058, 1060 may vary from the relatively large gap shown in FIG. 16 to a small gap or separation. Each coil 1056, 1058, 1060 may contain a number of windings of conductive wire 1064 that is contained within a non-conductive and non-magnetic housing 1066. In this embodiment, the various coils 1056, 1058, 1060 are dimensioned such that patient 1008 fits inside the interior portion 1061. For example, the coils 1056, 1058, 1060 may be dimensioned such that they can fit around a patient 1008 with a diameter of around 600 mm (23.6 inches). The magnetic sensor 1020 of FIGS. 16 and 17 is sized to be worn or donned by the patient 1008 during the measurement procedure. FIG. 16 illustrates a cross-sectional view illustrating how the coils 1056, 1058, 1060 are positioned about the body of the patient 1008. Also, the magnetic sensor 1020 is positioned such that the fluid 1002 located within the bulge region 1012 is located between the transmission coil 1056 and one of the receive coils 1058, 1060. For example, the magnetic sensor 1020 is positioned such that the fluid 1002 located within the bulge region 1012 is substantially centered in relation to the transmission coil 1056 and equidistant from the receive coils 1058, 1060. The transmission coil 1056 and the receive coils 1058, 1060 may also be formed from multiple conductors or wires having detachable couplings or electrical contacts that permit the same to act as a continuous wire. The transmission coil 1056 and the receive coils 1058, 1060 may also be integrated into a substrate or the like such as a flex circuit. This configuration would, for example, facilitate a design that allows a coil to be open and closed around a patient in a clamshell fashion.

As best seen in FIG. 17, a source of alternating current 1024 is connected to the transmission coil 1056. The transmission coil 1056 is also coupled to drive circuitry as explained herein that is used to drive or power the transmission coil 1056 with the alternating current. The two receive coils 1058, 1060 are disposed on either side and are connected to one another via, for example, wire or conductor 1062. Both receive coils 1058, 1060 are also coupled via wires or conductors 1068 to circuitry (not shown) for sensing electrical signals produced in response to the presence of the fluid 1002 in proximity to the magnetic sensor 1020. Typically, during operation, the magnetic sensor 1020 is designed such that in the absence of any magnetically detectable fluid 1002 a null or zero signal is produced by the two receive coils 1058, 1060. In this regard, the magnetic sensor 1020 is balanced or calibrated such that in the presence of the magnetically detectable fluid 1002, a signal is generated in the receive coils 1058, 1060 that is then picked up and process by sense electronics. Generally, the signal produced by the receive coils 1058, 1060 is proportional to the mass of the fluid 1002 and the position of the fluid 1002. For example, a larger mass of fluid 1002 will generally produce a larger signal than a small mass of fluid 1002. Similarly, a mass of fluid 1002 that is closer to the magnetic sensor 1020 will produce a larger signal than a mass of fluid 1002 that is further away from the magnetic sensor 1020.

In one preferred aspect, the magnetic sensor 1020 of FIGS. 16 and 17 is able to directly determine the quantity of fluid 1002 that is consumed by the patient 1008 during the test procedure. That is to say, the magnetic sensor 1020 is able to directly ascertain the volume or mass of fluid 1002 that exists within the bulge portion 1012 of the patient's stomach 1006. For example, by knowing or estimating the position of the bulge 1012 that will receive the liquid, the detected signal can be used to then determine the mass (or volume) of the fluid 1002 that is contained in the bulge 1012 after consumption. As explained herein, the quantity of fluid 1002 can be monitored over time to then determine the flow rate of the fluid 1002 through the stoma formed in the restricted portion 1004. In another aspect, the quantity of the fluid 1002 that is consumed may be known in advance. The progression of the signal as a function of time may then be correlated to determine the flow rate of the fluid 1002 through the stoma. It should be appreciated that the flow rate of the fluid 1002 may be determined based on empirical data showing the evolution of the detected signal over time or the flow rate of the fluid 1002 may be calculated directly using one or more known parameters (e.g., signal, and position of fluid 1002, etc.). The use of the single transmission coil 1056 and the two outer receive coils 1058, 1060 operates in a somewhat similar manner to linear variable differential transformer (LVDT) sensors which are typically used for measuring linear displacement.

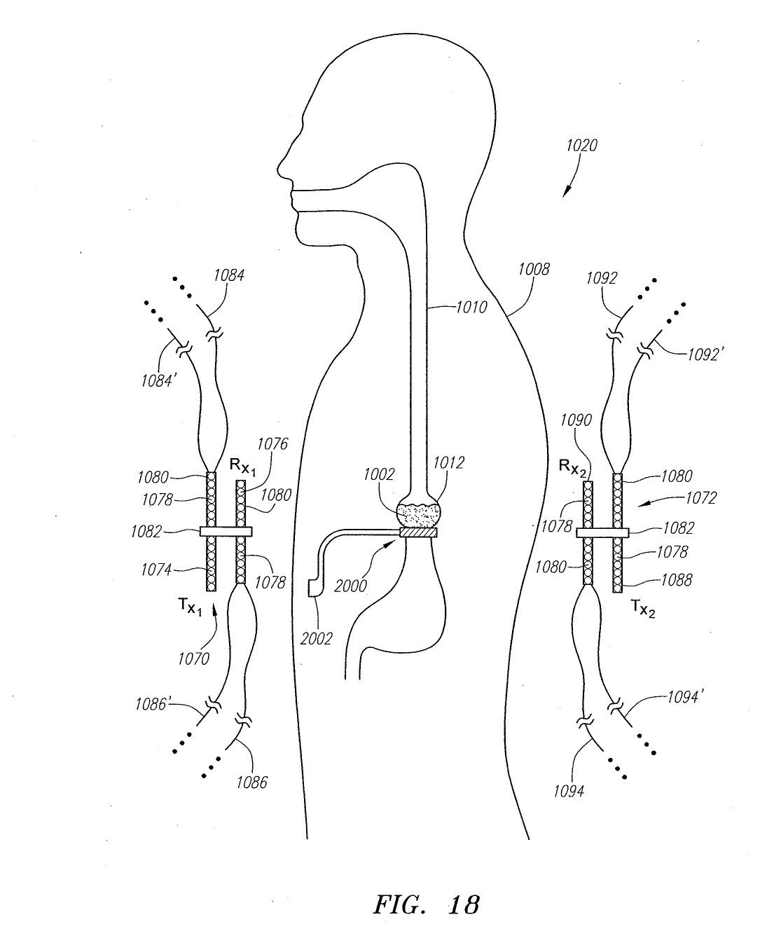

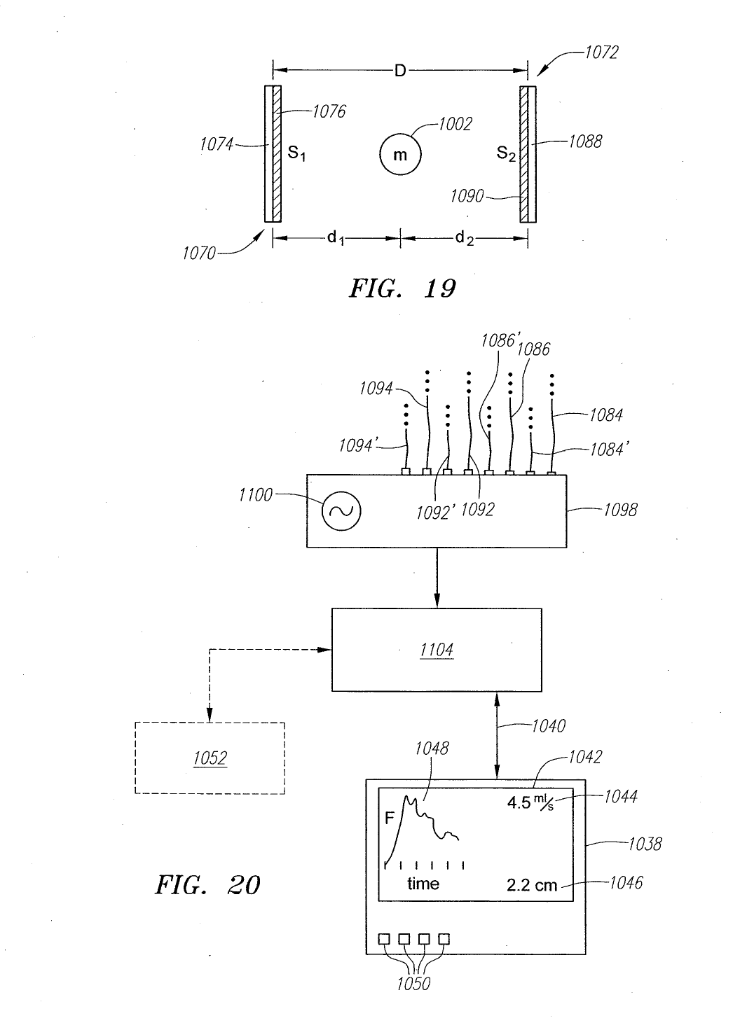

FIGS. 18 and 19 illustrate another embodiment of a magnetic sensor 1020. In this embodiment, two sets 1070, 1072 of transmit and receive coils are used to “triangulate” on the fluid 1002 contained in the bulge portion 1012. As best seen in FIG. 18, the first set of coils 1070 includes a transmission coil (Tₓ₁) 1074 and a receive coil (Rₓ₁) 1076. These coils 1074, 1076 may be formed by winding a conductor 1078 (e.g., wire or the like) in circular or spiral configuration. The conductor 1078 may be contained within a housing 1080. The coils 1074, 1076 may be mounted in a concentric manner or they may be partially offset from one another as illustrated in FIG. 18, for example, mounted on center shafts 1082. The transmission coil (Tₓ₁) 1074 and a receive coil (Rₓ₁) 1076 include respective signal lines 1084, 1084′ and 1086, 1086′. The second set of coils 1072 also include a transmission coil (Tₓ₂) 1088 and a receive coil (Rₓ₂) 1090. These coils 1088, 1090 may be formed by winding a conductor 1078 (e.g., wire or the like) in circular or spiral configuration. The conductor 1078 may be contained within a housing 1080. The coils 1088, 1090 may be mounted in a concentric manner or they may be partially offset as shown, for example, mounted on center shafts 1082. Of course, the shafts 1082 are not required and other mounting arrangements between the two sets of coils 1070, 1072 are contemplated by the invention. The transmission coil (Tₓ₂) 1088 and a receive coil (Rₓ₂) 1090 include respective signal lines 1092, 1092′ and 1094, 1094′.

In the embodiment illustrated in FIGS. 18 and 19, the quantity of fluid 1002, which may be represented by a mass or volume, can be mathematically derived by knowing the distance (D) between the two sets of coils 1070, 1072. In addition, the multi-coil embodiment described herein offers the advantage that the detected magnetic signal in response to the fluid 1002 does is not affected by the location of fluid 1002 as it moves closer or further away from the sets of coils 1070, 1072. For example, as the patient breaths or coughs which may move the fluid 1002 closer to one set of coils 1070, 1072, the combined signal from the two sets of coils 1070, 1072 maintains a relatively constant value so as to avoid unwanted perturbations in the signal.

With reference to FIG. 19, the signal (S₁) from the first set of coils 1070 is generally proportional to the mass (m) of the fluid 1002 as well as the distance (d₁) to the first set of coils 1070. This may be approximately expressed as follows where c₁ represents a constant or factor that is empirically derived or determined based on the configuration of the magnetic sensor 1020:

S₁=c₁d₁m Eq. 5

While the signal S₁ is linear near the center, the signal S₁ may not be linear as one gets closer to the coils 1070, 1072. For this reason, the signal S₁ may expressed as a power function or the like (e.g., S₁=c₁d₁^{a}m^{b}+c₂d₁^{c}m^{d}+c₃d₁^{e}m^{f}+ . . . ). Similarly, the signal (S₂) from the second set of coils 1072 is generally proportional to the mass (m) of the fluid 1002 as well as the distance (d₂) to the second set of coils 1072. This may be expressed as follows:

S₂=c₁d₂m Eq. 6

In this embodiment, the total distance (D) between the first set of coils 1070 and the second set of coils 1072 is known and kept constant during the procedure (d₁+d₂=D). Given the above relationships, the mass of the fluid 1002 can then be calculated as follows:

$$m = \frac{1}{{c}_{1} D} ( {S}_{1} + {S}_{2} ) \qquad Eq . 7$$

As seen in Equation 7 above, the mass of the fluid 1002 is based on the sum of the signals S₁ and S₂. If the mass of fluid 1002 were to move toward or away from one of the sets 1070, 1072 of coils one signal would decrease while the other would increase. Because the distance between the two sets of coils 1070, 1072 remains constant and the sum of the signals S₁, S₂ is used, the determined mass remains substantially constant. Of course, as the mass of fluid 1002 moves through the restricted portion 1004 of the gastric lumen 1006 (generally perpendicular to the face of the opposing sets of coils 1070, 1072), the measured mass (m) decreases. This decrease in mass, which may be converted to a volume given the density of the fluid 1002, can then be used to determine the real time flow rate of the fluid 1002 through the restricted portion 1004.

FIG. 20 illustrates further aspects of the embodiment illustrated in FIGS. 18 and 19. As seen in FIG. 20, the signal lines 1084, 1084′ and 1086, 1086′ from the first set of coils 1070 are input to a controller 1098. Similarly, the signal lines 1092, 1092′ and 1094, 1094′ from the second set of coils 1072 are input to the controller 1098. The controller 1098 may have integrated therein a source of alternating electrical current 1100. Of course, the source of alternating electrical current 1100 may be provided separately. The controller 1098 contains the circuitry for driving the first transmission coil 1074 and the second transmission coil 1088. Analog signals from the two receive coils 1076 and 1090 are input to the controller where they may be conditioned and amplified. The analog signals are then converted to digital signals which are then processed by processor 1104. The processor 1104 may include one or more dedicated processors or it may be, for example, a computer such as a personal or laptop computer loaded with appropriate software. The processor 1104 includes, for example, timing circuitry that is used to calculate the flow rate of the fluid 1002 over a period of time. The processor 1104 is also used to calculate the mass (m) as well other parameters such as flow rate which can then be reported to the user via the display 1038.

As seen in FIG. 20, the display 1038 may include a screen 1042 that can be used to display various parameters including, for example, the flow rate 1044 of the fluid 1002 through the stoma, the current size 1046 of the restriction device 2000, or a trace 1048 of an operating parameter (e.g., mass, volume, or flow rate) as a function of time. The display 1038 may also include one or more input devices 1050 as described herein. FIG. 20 also illustrates in phantom an optional external adjustment device 1052 that may be coupled to the processor 1104. As explained herein, the external adjustment device 1052 may be used to automatically adjust the gastric restriction device 2000 in a non-invasive manner. Of course, the external adjustment device 1052 is optional and manual adjustments may be made to the gastric restriction device 2000 to achieve the desired flow rate.

In one aspect of the invention, the flow rate of fluid 1002 through the restricted portion 1004 of the gastric lumen 1006 is calculated by the providing the magnetic sensor 1020 external to the patient 1008. The magnetic sensor 1020 may be donned by the patient 1008 such as illustrated in FIGS. 16 and 18. Alternatively, the magnetic sensor 1020 placed in relatively close proximity to the patient 1008. The magnetic sensor 1020 may physically touch the patient at one or more locations or, alternatively, the magnetic sensor 1020 may be disposed some distance away from the patient 1008. For example, the magnetic sensor 1020 may be integrated into chair with, for example, one set of coils 1070 is located in the back of the chair while another set of coils 1072 are brought against or adjacent to the abdomen of the patient 1008. For example, the set of coils 1072 may be positioned on a moveable arm or the like that can be swung into position after the patient sits down within the chair. Alternatively, the first and second sets of coils 1070, 1072 may be positioned on a cart or other device in which the patient 1008 can be positioned. In still another aspect, one of the sets of coils 1070, 1072 may be posited on a vertical surface such as a wall while the remaining set of coils 1070, 1072 may be moved into position on an opposing side of the patient 1008. These configurations allow testing to be performed with patient in a substantially vertical, seated or standing position. This best simulates typical conditions during eating.

The patient 1008 then consumes the magnetically detectable fluid 1002. The fluid 1002 may be a known quantity (e.g., 25 ml) or, alternatively, the patient 1008 may consume an unknown quantity of fluid 1002. After consumption, the quantity of fluid 1002 that is disposed upstream of the restricted portion 1004 of the gastric lumen 1006 (e.g., in the bulge portion 1012) is measured by the magnetic sensor 1020. In one aspect of the invention, prior to performing the flow tests, the gastric restriction device 2000 may be adjusted to produce a fully closed stoma which can then be slowly opened to increase flow as measurements are taken. In this regard, after the patient 1008 consumes the fluid 1002, substantially all of the fluid is disposed upstream of the restricted portion 1004. Alternatively, however, the flow test may be performed at the current setting of the gastric restriction device 2000. One advantageous benefit is that the tissue of the human body does not have significant magnetic properties that would have any confounding affect on the inventive systems of FIGS. 15 through 18. Therefore, any dynamic changes in the tissue of the stomach wall at the stoma, for example during adjustment of the restriction device, do not significantly change or affect the measurements.

The magnetic sensor 1020 then measures the quantity of fluid disposed upstream of the restricted portion 1004 as time progresses. For example, the magnetic sensor 1020 may sample or detect the quantity of fluid 1002 on a periodic basis. In one aspect, the quantity of fluid 1002 may be measured with a frequency of 2 Hz or higher. Assuming a partially or fully opened stoma, the quantity of fluid 1002 measured at later time intervals generally decreases as the fluid 1002 passes through the restricted portion 1004. The flow rate can then be determined by subtracting the quantity of fluid 1002 obtained at two different times and dividing this number by the elapsed time between when these measurements were made. Flow measurements may be obtained in real time when measurements are made on a frequent basis.

The difference or change in quantity of fluid 1002 that is measured may be between successive time intervals or, alternatively, may be determined over a time interval that spans over multiple measurement cycles. This later method may be chosen to average out the results or to reduce variability in measurements. For instance, a rolling or moving average might be calculated that is based on the last “x” number of readings obtained from the magnetic sensor 1020.

The system 1000 described with respect to FIGS. 15-20 is advantageous because the physician or other skilled technician is able to use the magnetic sensor 1020 to determine the actual or real time flow rate of fluid 1002 through a restricted stoma. While other methods may permit the calculation of a bulk or average flow rate from the complete passing of a fluid through a restriction, these methods have been unable to discern the real time flow rates that are occurring through the restricted stoma. Not even barium consumption in combination with x-ray fluoroscopy can provide real time feedback, because there is no known way to visually quantify, with accuracy, a partially passed volume of barium through the restricted stoma. Physicians and others are interested in obtaining real time flow rate data because it more accurately reflects the behavior of fluid passing through the restricted stoma.

Fluid or food does not typically pass through the stoma at a steady rate. Peristaltic contractions typically cause an intermittent or periodic flow rate reading if assessing the flow rate in real time. The peak flow rate during this period can be an indicator of the effect of a tight restriction. For example the likelihood of esophageal dilatation may possibly be predicted by determining the peak flow rate. The non-invasive method described herein is less invasive than esophageal pressure measurements, during which a pressure measurement catheter or probe is placed directly into the patient's esophagus 1010. In addition to the peak flow rate, the frequency or consistency of the peristaltic contractions (i.e., the number of contractions per time) can also be easily and non-invasively determined. By identifying typical patterns of test flow traces, patients 1008 may be able to be grouped by severity of esophageal condition or by peristaltic pattern, to help determine not only how tightly their restriction should be adjusted, but also, for example, whether a more conservative diet should be selected.

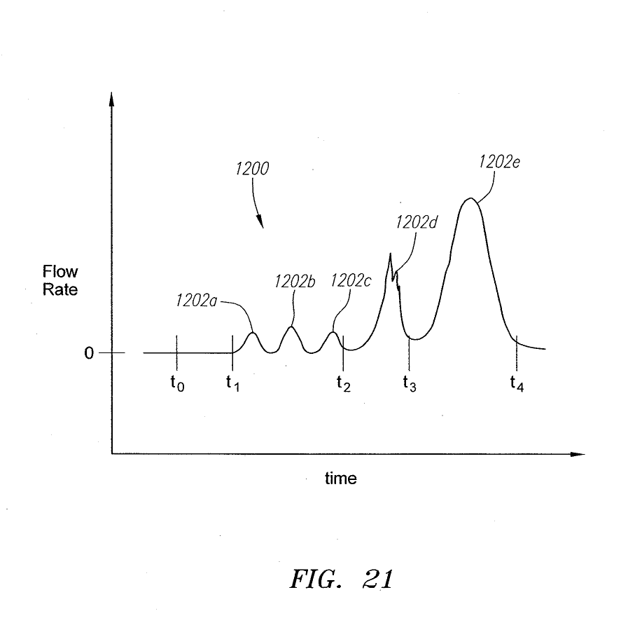

In addition, the peristaltic phenomenon may be used in conjunction with the real time flow measurement. For example, during one type of dynamic adjustment, the restriction device is tightened completely, causing complete occlusion at the stoma. Then the restriction device is slowly loosened until the desired stoma size is reached. Current methods are very inconsistent in achieving the desired results with this method. By assessing a group of several peristaltically-driven pulses, a better comparison between different degrees of stoma tightness can be more easily compared, without the need for the patient to ingest a large amount of test fluid 1002. For example, FIG. 21 illustrates a real time flow rate trace 1200 having a plurality of peristaltic pulses 1202a, 1202b, etc. At time to, the stoma is completely restricted. At time t₁, the stoma is loosened enough such that flow begins through the stoma, as seen in pulses 1202a, 1202b and 1202c. From times t₂ to t₃ a second loosening is performed. During this time period, the pulse 1202d is too dynamic to be easily compared to the first three pulses, 1202a, 1202b, 1202c, due to the increase in flow due caused by the loosening. However, the subsequent pulse 1202e, occurs from time t₃ to t₄ during a period completely after the loosening. The area under the curve 1202c can be compared with the area under the curve 1202e by the processor 1104 and the peaks can also be compared, in order to more accurately compare the effect of the second loosening without interference from the loosening act itself.

It should be understood that the patient 1008 has only swallowed a single portion of the test fluid 1002, and the desired adjustment point does not need to be found by trial and error, which would require several portions or aliquots of the test fluid 1002. The processor 1104 can be configured to look for a specific difference between the pre-adjustment pulse 1202c and the post-adjustment pulse 1202e, and to ignore completely the during adjustment pulse 1202d. This can be achieved from an output signal of the gastric restriction device 2000 or from the adjustment device 1052 that is sent to the processor 1104, and thus determines which pulses will be examined. When the desired characteristic of pulse 1202e is above the desired threshold (for example peak flow rate or average flow rate or area under the curve (volume/pulse)), the processor 1104 indicates (for example with a beep or other signal) that the adjustment is adequate.

Other embodiments are contemplated and are considered to fall within the scope of this invention. For example, in any of the embodiments, a single coil may be used as both the transmit coil 1022 and the receive coil 1030. The single coil may be operated by a controller 1026 or 1098 so that transmit pulses are timed to alternate with received pulses. This allows a simpler configuration, with fewer actual coils. Using this methodology, the four coil configuration of FIG. 18 could be accomplished using only two coils and the two coil configuration of FIG. 15 could be accomplished using only a single coil.

Thus, the present disclosure is not meant to be limited in scope by the exemplary embodiments described herein, which are intended as single illustrations of individual aspects of the disclosure. As a result, it is intended that functionally equivalent methods and components are within the scope of the disclosure. Indeed, various modifications of the disclosure, in addition to those shown and described herein will become apparent to those skilled in the art, and all such modifications and variations are intended to fall within the scope of the disclosure. For example, while the embodiments presented herein have provided examples in terms of gastric restriction devices, it is contemplated that embodiments can be provided that analyze fluid movement past a restriction of the lumen of any passage through which a substance is flowing.

1. A method for determining the flow rate of a fluid passing through a restricted portion of a gastric lumen of a patient comprising:
providing a magnetic sensor external to the patient, the magnetic sensor configured to detect a quantity of fluid disposed upstream of the restricted portion of the gastric lumen;
measuring the quantity of fluid disposed upstream of the restricted portion of the gastric lumen at a first time with the magnetic sensor;
measuring the quantity of fluid disposed upstream of the restricted portion of the gastric lumen at a second time with the magnetic sensor; and
determining the flow rate by subtracting the quantity of fluid disposed upstream of the restricted portion of the gastric lumen at the second time from the quantity of fluid disposed upstream of the restricted portion of the gastric lumen at the first time and dividing the subtracted value by the elapsed time between the first time and the second time.

2. The method of claim 1, wherein the elapsed time between the first time and the second time is less than 0.5 seconds.

3. The method of claim 1, further comprising displaying the determined flow rate on a display operatively coupled to the magnetic sensor.

4. The method of claim 1, wherein the restricted portion of the gastric lumen comprises a portion of the patient's stomach at least partially restricted by a gastric restriction device.

5. The method of claim 1, wherein the quantity of fluid disposed upstream of the restricted portion of the gastric lumen at the first time is obtained after the patient consumes a known volume of fluid.

6. The method of claim 1, wherein the quantity of fluid disposed upstream of the restricted portion of the gastric lumen at the first time is obtained after the patient consumes an unknown volume of fluid.

7. The method of claim 1, further comprising measuring the quantity of fluid disposed upstream of the restricted portion of the gastric lumen at a third time and determining the flow rate by subtracting the quantity of fluid disposed upstream of the restricted portion of the gastric lumen at the third time from the quantity of fluid disposed upstream of the restricted portion of the gastric lumen at the second time and dividing the subtracted value by the elapsed time between the third time and the second time.

8. The method of claim 1, wherein the measured quantity of fluid disposed upstream of the restricted portion of the gastric lumen at the second time is zero.

9. A method of claim 1, wherein the fluid comprises a mixture of magnetic material contained within a carrier fluid.

10. A method of claim 9, wherein the magnetic material comprises a plurality of particles.

11. A method of claim 1, wherein the fluid comprises a mixture of conductive material contained within a carrier fluid.

12. The method of claim 11, wherein the conductive material comprises a plurality of particles.

13. The method of claim 9, wherein the magnetic material comprises magnetite.

14. The method of claim 9, wherein the magnetic material comprises a gadolinium compound.

15. A method of adjusting a gastric restriction device configured to restrict a portion of a gastric lumen of a patient comprising:
administering a volume of test fluid to a patient, the test fluid comprising a carrier fluid and a magnetically detectable component;
measuring the flow rate of the test fluid through the restriction of the gastric lumen using a magnetic sensor device located external to the patient; and
adjusting the gastric restriction device to achieve a target flow rate through the restriction.

16. The method of claim 15, wherein the gastric restriction device is manually adjusted in response to the measured flow rate.

17. The method of claim 15, wherein the gastric restriction device is automatically adjusted in response to the measured flow rate.

18. The method of claim 15, wherein the flow rate through the restriction is measured at least in part by comparing the quantity of test fluid located upstream of the restriction at a plurality of different times.

19. The method of claim 15, wherein the flow rate of test fluid through the restriction is measured repeatedly.

20. The method of claim 19, wherein the gastric restriction device is adjusted between flow rate measurements.

21. The method of claim 15, wherein the magnetic sensor device comprises a transmit coil and a receive coil.

22. The method of claim 15, wherein the magnetic sensor device is configured to move adjacent to an external surface of the patient.

23. A method of determining the quantity of fluid within a gastric lumen of the patient comprising:
administering a quantity of test fluid to a patient, the test fluid comprising a carrier fluid and a magnetically detectable component; and
measuring the quantity of test fluid contained in the gastric lumen using a magnetic sensor device located external to the patient.

24. The method of claim 23, wherein the test fluid is contained in the patient's stomach.

25. The method of claim 23, wherein the test fluid is contained in the patient's esophagus.

26. A system for determining the flow rate of fluid passing through a restricted portion of a gastric lumen of a patient comprising:
at least one transmit coil operatively coupled to an alternating current source; and
at least one receive coil configured for detecting a quantity of a magnetically detectable fluid contained in the gastric lumen, the at least one receive coil operatively coupled to a controller configured to output a signal corresponding to the quantity of magnetically detectable fluid contained in at least a portion of the gastric lumen over a period of time.

27. The system of claim 26, wherein the at least one transmit coil comprises two transmit coils and the at least one receive coil comprises two receive coils.

28. The system of claim 26, wherein the at least one transmit coil and the at least one receive coil comprise the same coil.

29. The system of claim 26, further comprising a display operatively coupled to the controller.

30. The system of claim 29, wherein the display provides the user with an indication of a mass per unit of time of the magnetically detectable fluid that passes through the gastric lumen.

31. The system of claim 29, wherein the display provides the user with an indication of the volume per unit of time of the magnetically detectable fluid that passes through the gastric lumen.

32. The system of claim 26, further comprising an external adjustment device operatively coupled to the controller, the external adjustment device being configured to adjust a gastric restriction device disposed about the restricted gastric lumen of the patient.

33. The system of claim 32, wherein the controller automatically adjusts the gastric restriction device via the external adjustment device in response to the output signal corresponding to the quantity of magnetically detectable fluid contained in the gastric lumen.

34. The system of claim 33, wherein adjustment of the gastric restriction device is stopped once a target flow rate of the magnetically detectable fluid is reached.

35. The system of claim 26, wherein the magnetically detectable fluid comprises a mixture of magnetic material contained within a carrier fluid.

36. The system of claim 35, wherein the magnetic material comprises a plurality of particles.

37. The system of claim 35, wherein the magnetic material comprises magnetite.

38. The system of claim 35, wherein the magnetic material comprises a gadolinium compound.

39. The system of claim 26, wherein the magnetically detectable fluid comprises a mixture of conductive material contained within a carrier fluid.

40. The system of claim 39, wherein the conductive material comprises a plurality of particles.

41-44. (canceled)
